SEQUENCE LISTING (3 sequences)

SEQ ID NO: 1 - DNA, 22 bp, Artificial Sequence
  PNA capture molecule
aaccatacaacctactacctca

SEQ ID NO: 2 - DNA, 22 bp, Artificial Sequence
  analyte
tgaggtagtaggttgtatggtt

SEQ ID NO: 3 - DNA, 36 bp, Artificial Sequence
  analyte
ggaagggagtaaagttaatacctttgctcattgacg

(19) United States
(12) Patent Application Publication
(10) Pub. No.: US 2010/0285601 A1
(43) Pub. Date: Nov. 11, 2010

(54) METHOD OF ELECTRICALLY DETECTING A NUCLEIC ACID MOLECULE

(75) Inventors: Jinming Kong, Singapore (SG); Yi Fan, Singapore (SG); Xiantong Chen, Singapore (SG)

Correspondence Address: INTELLECTUAL PROPERTY / TECHNOLOGY LAW, PO BOX 14329, RESEARCH TRIANGLE PARK, NC, 27709, US

(73) Assignee: AGENCY FOR SCIENCE, TECHNOLOGY AND RESEARCH, (SG)

(21) Appl. No.: 12/680,290

(22) PCT Filed: Sep. 28, 2007
(86) PCT No.: PCT/SG07/00330

§ 371 Date: Jul. 20, 2010

(51) Int. Cl. G01N 27/00

(52) U.S. Cl. 436/94; 422/82.02

(57) ABSTRACT

The method is performed by means of a pair of electrodes that are arranged at a distance and within a sensing zone. A nucleic acid capture molecule with an uncharged backbone and a nucleotide sequence that is at least partially complementary to at least a portion of a strand of the target nucleic acid molecule, is immobilised on an immobilisation unit. The immobilisation unit, which is arranged within the sensing zone, is contacted with a solution suspected to comprise the target nucleic acid molecule, which hybridizes to the nucleic acid capture molecule. An activation agent is added, which has an electrostatic net charge complementary to the net charge of the target nucleic acid molecule. It associates to the complex of nucleic acid capture molecule and target nucleic acid molecule. Added is a water soluble polymer with at least one polymer strand and with an electrostatic net charge that is complementary to the net charge of the activation agent. Thus the polymer associates to the activation agent. A metal salt is added, which can act as an oxidant and the metal ions of which have an electrostatic net charge complementary to the net charge of the polymer. The metal ions associate to the polymer. Upon adding a reducing agent, the latter reduces the metal ions, forming a metal wire. The presence of the analyte molecule is determined based on an electrical characteristic of a region in the sensing zone that is affected by the metal wire.

### FIELD OF THE INVENTION

The present invention relates to a method of electrically detecting a nucleic acid molecule, in particular detecting the nucleic acid molecule by means of an electrode pair.

### BACKGROUND OF THE INVENTION

The detection of nucleic acids is an important method not only in analytical chemistry but also in biochemistry, food technology, forensic technology, agriculture and medicine. It is for example a critical factor in diagnostics of genetic, bacterial, and viral diseases and environmental micro-organisms monitoring. The most frequently used methods for determining the presence and concentration of nucleic acids include the detection by autoradiography, fluorescence, chemiluminescence or bioluminescence as well as electro-chemical and electrical techniques (for an overview see Odentahl, K. J. & Gooding, J. J., Analyst (2007) 132, 603-610). Typical nucleic acid detection techniques rely on marking of the nucleic acid to be examined. The molecular markers used for this purpose may be fluorescent, luminescent or electrically or magnetically active molecules or particles (quantum dots, magnetic beads). After a hybridization e.g. with complementary DNA capture molecules in a homogeneous phase or at a microarray, the markers are detected at the bound nucleic acids by one of the aforementioned methods. Electrical and electrochemical biosensors allow for fast and real-time analysis. Electrical techniques include conductivity measurements, which can for instance be based on an oligonucleotide functionalised with a gold nanoparticle (Park, S. J., et al., Science (2002) 295, 1503-1506) or with a conductive polymer (US patent application 2005/0079533). In many detection methods the binding of an analyte to the capture probe changes the conductivity or other electrical properties between two electrodes. In other biosensors a field effect transistor (FET), such as an ion-sensitive field effect transistor (ISFET) is used, for instance by modifying the gate electrode or by immobilising a capture probe thereon (see Schöning, M. J. & Poghossian, A., Analyst (2002) 127, 1137-1151 for a review).

The basic elements for detection and quantification of nucleic acids by using analytical devices, whether optical, electrochemical, or other techniques are selected, are still a challenge in terms of accuracy, sensitivity, selectivity, simplicity and practicality. Advances in nanotechnology, especially in nanoparticles and nanowires, have showed a great potential and paved a new avenue for addressing these issues.

In the past years the great potential of metal nanoparticles as an alternative label for ultra-sensitive detection of nucleic acids has been shown. By resorting to localizing gold nanoparticles in an electrode gap ensuing with silver deposition facilitated by these nanoparticles bridges the gap, Park et al (Science (2002) 295, 1503-1506) demonstrated readily measurable conductivity changes (see FIG. 1). The array-based electrical detection of DNA on a chip of inter-digital electrode structure was reported to detect target DNA at concentrations as low as 500 femtomolar with a point mutation selectivity factor of approximately 100,000:1. Braun et al. (Nature (1998) 391, 775-778) fabricated a DNA-template-based silver nanowire around 100 nm thick and 15 mm long. By fixing λ-DNA in between two electrical contacts and activating the λ-DNA with an AgNO₃ basic aqueous solution, silver ions bind to the DNA skeleton. The DNA templated wire was metallized with silver ions by hydroquinone reduction under low light conditions. The resulting nanowires showed a resistance of several mega-ohms as a result of the coagulation of silver, which forms clusters with a diameter of 50 nm.

Further important advances in the means of metal nanowire formation were achieved, such as a thin continuous palladium film onto single DNA molecules (Richter, J., et al., Applied Physics Letters (2001) 78, 4, 536-538), DNA templated platinum nanowire (Mertig, M., et al., Nano Letters (2002) 2, 2, 841-844), Cu metal onto surface-attached DNA (Monson, C. F., & Woolley, A. T., Nano Letters (2003) 3, 3, 359-363), Au-nanoparticle nanowires based on DNA and polylysine templates (Patolsky, F., Y et al., Angew. Chem. Int. Ed. (2002) 41, 13, 2323-2327). The intra- and intermolecular recognition properties of nucleic acids have also been used to build-up nanostructures (Seeman, N. C., Nature (2003) 421, 33-37; Glidle, A., et al., Nano Letters (2006) 6, 3, 445-448). A DNA detection based on capacitance by means of a metal nanowire showed a sensitivity down to 0.2 nM (Moreno-Hagelsieb, L., et al., Sens. Actuat. B-Chem. (2004) 98, 269-274).

Nevertheless, current electrical detection approaches either have low sensitivity or required labelling nucleic acids with gold nanoparticles. Procedures such as gold nanoparticle preparation and functionality are tedious and require highly skilled techniques. Thus, there remains a need for an alternative method for the detection of nucleic acids.

Accordingly it is an object of the present invention to provide a method of electrically detecting a nucleic acid molecule, which avoids these disadvantages.

### SUMMARY OF THE INVENTION

According to a first aspect, the invention provides a method for electrically detecting a biological analyte molecule by means of a pair of electrodes. The electrodes are arranged at a distance from one another. Further, the pair of electrodes is arranged within a sensing zone. The method includes immobilising on an immobilisation unit a nucleic acid capture molecule. The nucleic acid capture molecule has an at least essentially uncharged backbone. It further has a nucleotide sequence that is at least partially complementary to at least a portion of a strand of the target nucleic acid molecule. The immobilisation unit is arranged within the sensing zone. The method also includes contacting the immobilisation unit with a solution suspected to include the target nucleic acid molecule. Furthermore, the method includes allowing the target nucleic acid molecule to hybridise to the nucleic acid capture molecule on the immobilisation unit. Thereby the method includes allowing the formation of a complex between the nucleic acid capture molecule and the target nucleic acid molecule. The method further includes adding an activation agent. The activation agent has an electrostatic net charge that is complementary to the electrostatic net charge of the target nucleic acid molecule. As a result, the activation agent associates to the complex formed between the nucleic acid capture molecule and the target nucleic acid molecule. The method also includes adding a water soluble polymer. The water soluble polymer has an electrostatic net charge that is complementary to the electrostatic net charge of the activation agent. Thereby the method includes allowing the water soluble polymer to associate to the activation agent that is associated to the complex between the nucleic acid capture molecule and the target nucleic acid molecule. The method also includes adding a metal salt. The metal salt is capable of acting as an oxidant. Further, the metal ions of the metal salt having an electrostatic net charge that is complementary to the electrostatic net charge of the water soluble polymer. Thereby the method includes allowing the metal ions to associate to the water soluble polymer. As a result a plurality of metal ions covers the surface of the water soluble polymer. The method also includes adding a reducing agent. Thereby the method includes allowing the reducing agent to reduce the metal ions of the metal salt to the corresponding metal. As a result a metal wire is formed from the plurality of metal ions covering the surface of the polymer. Furthermore the method includes determining the presence of the target nucleic acid molecule based on an electrical characteristic of a region in the sensing zone. The electrical characteristic is influenced by the electrical conductivity of the metal wire.

According to a second aspect the invention provides a kit for electrically detecting a target nucleic acid molecule. The kit includes a pair of electrodes. The electrodes are arranged at a distance from one another. Further, the pair of electrodes is arranged within a sensing zone. The kit also includes an immobilisation unit. The immobilisation unit is arranged within the sensing zone. The kit further includes a nucleic acid capture molecule. The nucleic acid capture molecule has an at least essentially uncharged backbone. Further the nucleic acid capture molecule has a nucleotide sequence that is at least partially complementary to at least a portion of the target nucleic acid molecule. The kit also includes an activation agent. The activation agent has an electrostatic net charge that is complementary to the electrostatic net charge of the target nucleic acid molecule. Furthermore, the kit includes a water soluble polymer. The water soluble polymer has an electrostatic net charge that is complementary to the electrostatic net charge of the activation agent. The kit also includes a metal salt. The metal salt is capable of acting as an oxidant. The metal ions of the metal salt have an electrostatic net charge that is complementary to the electrostatic net charge of the water soluble polymer. The kit also includes a reducing agent. The reducing agent is capable of reducing the metal ions of the metal salt to the corresponding metal.

### BRIEF DESCRIPTION OF THE DRAWINGS

The invention will be better understood with reference to the detailed description when considered in conjunction with the non-limiting examples and the accompanying drawings.

FIG. 1 depicts a schematic representation of electrical detection of DNA hybridization using gold nanoparticle labels and gold enhancement according to a conventional method.

FIG. 2 depicts a schematic representation of a method according to the invention, in which a nucleic acid capture molecule (1) is immobilised on an immobilisation unit (5) and forms a complex with the target nucleic acid molecule (2). Upon addition of an activation agent (Zr⁴⁺), a water soluble polymer (4), a metal salt (Cu²⁺) and a reducing agent (ascorbic acid) a metal wire is formed on the complex of the nucleic acid molecules.

FIG. 3 illustrates examples of different electrode arrangements relative to an immobilisation unit (5) on which a nucleic acid capture molecule (1) is immobilised. The examples are three ring-shaped electrodes (40) (FIG. 3A), an array of electrodes (3) (FIG. 3B) and two interdigital electrodes (50) (FIG. 3C, FIG. 3D in top view).

FIG. 4 depicts a schematic representation of a further electrode arrangement that can be used in a method of the invention, in which the nucleic acid capture molecule (1) is immobilised on the gate electrode (61) of a field effect transistor.

FIG. 5 depicts an electrode arrangement in which the nucleic acid capture molecule (1) is immobilised on an additional, electrically floating gate (64) of a field effect transistor.

FIG. 6 shows the resistance determined of a target nucleic acid being DNA, and a control (indistinguishable) at a concentration of 1×10⁻¹²M before copper deposition (left), a control after copper deposition (middle) and target DNA after copper deposition (right).

### DETAILED DESCRIPTION OF THE INVENTION

The present invention provides a method of the electrically detecting a nucleic acid molecule. As used herein, the term ‘detection’, ‘detecting’ or ‘detect’ refers broadly to measurements which provide an indication of the presence or absence, either qualitatively or quantitatively, of an analyte. Accordingly, the term encompasses quantitative measurements of the concentration of an analyte nucleic acid molecule in a sample, as well as qualitative measurements in which for instance different types of analyte molecules in a given sample are identified, or, as a further example, the behaviour of a particular analyte molecule in a given environment is observed. The term ‘quantification’ refers solely to quantitative measurements of the amount, e.g. the concentration, of an analyte molecule.

The term “nucleic acid molecule” as used herein refers to any nucleic acid in any possible configuration, such as single stranded, double stranded or a combination thereof. Nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA) and tecto-RNA molecules (e.g. Liu, B., et al., J. Am. Chem. Soc. (2004) 126, 4076-4077). LNA has a modified RNA backbone with a methylene bridge between C4′ and O2′, providing the respective molecule with a higher duplex stability and nuclease resistance. DNA or RNA may be of genomic or synthetic origin. A respective nucleic acid may furthermore contain non-natural nucleotide analogues and/or be linked to an affinity tag or a label.

Many nucleotide analogues are known and can be used in nucleic acids used in the methods of the invention. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

A target nucleic acid molecule that can be detected (including quantified) by the method of the present invention can originate from a large variety of sources. Samples that include or are suspected or expected to include the respective analyte molecule include biological samples derived from plant material and animal tissue (e.g. insects, fish, birds, cats, livestock, domesticated animals and human beings), as well as blood, urine, sperm, stool samples obtained from such animals. Biological tissue of not only living animals, but also of animal carcasses or human cadavers can be analysed, for example, to carry out post mortem tissue biopsy or for identification purposes, for instance. In other embodiments, samples may be water that is obtained from non-living sources such as from the sea, lakes, reservoirs, or industrial water to determine the presence of a targeted bacteria, pollutant, element or compound. Further embodiments include, but are not limited to, dissolved liquids or suspensions of solids. In yet another embodiment, quantitative data relating to the analyte is used to determine a property of the fluid sample, including analyte concentration in the fluid sample, reaction kinetic constants, analyte purity and analyte heterogeneity.

Accordingly, any of the following samples selected from, but not limited to, the group consisting of a soil sample, an air sample, an environmental sample, a cell culture sample, a bone marrow sample, a rainfall sample, a fallout sample, a sewage sample, a ground water sample, an abrasion sample, an archaeological sample, a food sample, a blood sample, a serum sample, a plasma sample, an urine sample, a stool sample, a semen sample, a lymphatic fluid sample, a cerebrospinal fluid sample, a nasopharyngeal wash sample, a sputum sample, a mouth swab sample, a throat swab sample, a nasal swab sample, a bronchoalveolar lavage sample, a bronchial secretion sample, a milk sample, an amniotic fluid sample, a biopsy sample, a cancer sample, a tumour sample, a tissue sample, a cell sample, a cell culture sample, a cell lysate sample, a virus culture sample, a nail sample, a hair sample, a skin sample, a forensic sample, an infection sample, a nosocomial infection sample, a production sample, a drug preparation sample, a biological molecule production sample, a protein preparation sample, a lipid preparation sample, a carbohydrate preparation sample, a space sample, an extraterrestrial sample or any combination thereof may be processed in a method of the invention. Where desired, a respective sample may have been pre-processed to any degree. As an illustrative example, a tissue sample may have been digested, homogenised or centrifuged prior to being used with the device of the present invention. The sample may furthermore have been prepared in form of a fluid, such as a solution. Examples include, but are not limited to, a solution or a slurry of a nucleotide, a polynucleotide, a nucleic acid, a peptide, a polypeptide, an amino acid, a protein, a synthetic polymer, a biochemical composition, an organic chemical composition, an inorganic chemical composition, a metal, a lipid, a carbohydrate or of any combinations thereof. Further examples include, but are not limited to, a suspension of a cell, a virus, a microorganism, a pathogen or of any combinations thereof. It is understood that a sample may furthermore include any combination of the aforementioned examples. As an illustrative example, the sample that includes the biological analyte molecule (e.g. nucleic acid molecule) may be a mammal sample, for example a human or mouse sample, such as a sample of total mRNA. The analyte, which may be suspected or known to be present within the sample, may also be termed the “target”, and accordingly an analyte molecule may be termed the “target molecule”.

In some embodiments the sample is a fluid sample, such as a liquid. In other embodiments the sample is solid. In case of a solid or gaseous sample, an extraction by standard techniques known in the art may be carried out in order to dissolve the biological analyte molecule in a solvent. Accordingly, the biological analyte molecule, or the suspected/expected biological analyte molecule, is provided in form of a solution for the use in the present invention. As an illustrative example, the biological analyte molecule may be provided in form of an aqueous solution.

If desired, further matter may be added to the respective solution, for example dissolved or suspended therein. As an illustrative example an aqueous solution may include one or more buffer compounds. Numerous buffer compounds are used in the art and may be used to carry out the various processes described herein. Examples of buffers include, but are not limited to, solutions of salts of phosphate, carbonate, succinate, carbonate, citrate, acetate, formate, barbiturate, oxalate, lactate, phthalate, maleate, cacodylate, borate, N-(2-acetamido)-2-amino-ethanesulfonate (also called (ACES), N-(2-hydroxyethyl)-piperazine-N′-2-ethanesulfonic acid (also called HEPES), 4-(2-hydroxyethyl)-1-piperazine-propanesulfonic acid (also called HEPPS), piperazine-1,4-bis(2-ethanesulfonic acid) (also called PIPES), (2-[Tris(hydroxymethyl)-methylamino]-1-ethansulfonic acid (also called TES), 2-cyclohexylamino-ethanesulfonic acid (also called CHES) and N-(2-acetamido)-iminodiacetate (also called ADA). Any counter ion may be used in these salts; ammonium, sodium, and potassium may serve as illustrative examples. Further examples of buffers include, but are not limited to, triethanolamine, diethanolamine, ethylamine, triethylamine, glycine, glycylglycine, histidine, tris(hydroxymethyl)aminomethane (also called TRIS), bis-(2-hydroxyethyl)-imino-tris(hydroxymethyl)methane (also called BIS-TRIS), and N-[Tris(hydroxymethyl)-methyl]-glycine (also called TRICINE), to name a few. The buffers may be or be included in aqueous solutions of such buffer compounds or solutions in a suitable polar organic solvent. One or more respective solutions may be used to accommodate the suspected biological analyte molecule as well as other matter used, throughout an entire method of the present invention.

Further examples of matter that may be added, include salts, detergents or chelating compounds. As yet a further illustrative example, nuclease inhibitors may need to be added in order to maintain a nucleic acid molecule in an intact state. While it is understood that for the purpose of detection any matter added should not obviate the formation of a complex between the capture molecule (such as a nucleic acid capture molecule including a PNA capture molecule, see below) and the biological analyte molecule, for the purpose of carrying out a control measurement a respective agent may be used that blocks said complex formation.

As an illustrative example, the presence of a certain DNA or RNA sequence can be detected using the present invention for identifying a disease state. A respective DNA or RNA may for instance be heterolog, e.g. bacterial or viral. Diseases which can be detected include communicable diseases such as Severe Acute Respiratory Syndrome (SARS), Hepatitis A, B and C, HIV/AIDS, malaria, polio and tuberculosis; congenital conditions that can be detected pre-natally (e.g. via the detection of chromosomal abnormalities) such as sickle cell anemia, heart malformations such as atrial septal defect, supravalvular aortic stenosis, cardiomyopathy, Down's syndrome, clubfoot, polydactyl), syndactyl), atropic fingers, lobster claw hands and feet, etc. The present method is also suitable for the detection and screening for cancer.

The method of the present invention allows detecting a target nucleic acid molecule by means of an electrode arrangement such as a pair of electrodes. The term “electrode” as used herein is employed in its conventional sense, thereby referring to an object that is capable of serving as an electric conductor, through which an electrical current or voltage may be brought into and/or out of a medium in contact with the electrode. Typically an electrode is one of at least two terminals of an electrically conducting medium. The term “electrode arrangement” or “pair of electrodes” as used herein refers to any number of electrodes of two or higher. Accordingly, two or more electrodes are provided in the method (as well as the kits, see below) of the invention. The electrodes are arranged at a distance from one another. In embodiments where two electrodes are provided, the two electrodes may for instance be separated by a gap. In such embodiments the two electrodes of this pair of electrodes may face each other across the gap. In some embodiments the two electrodes are at least essentially parallel. The electrodes may be of any desired dimension and shape. They may for example have the shape of a flat, arched, concave or convex slab. In some embodiments they may have the shape of a ring (for an example see Green, B. J, & Hudson, J. L., Phys. Rev. E (2001), 63, 026214). In some embodiments interdigital electrodes are provided, which typically include a digit-like or finger-like pattern of parallel in-plane electrodes (see Mamishev, A. V., Proc. IEEE (2004), 92, 5, 808-845, or Matsue, T., Trends Anal. Chem. (1993), 12, 3, 100-108 for examples). In some embodiments an array of electrodes may be provided. If desired, one or more floating electrodes may be used. In some embodiments the electrodes that are provided are of similar size, for example of identical size.

The distance between the two or more electrodes (to which is also referred herein as gap) may be of any dimension, as long as the change of an electrical characteristic of the respective region can be determined in the method of the present invention (see below), so that a detection of a target nucleic acid molecule can be carried out. In some embodiments where more than two electrodes are provided, the distance at which the electrodes are arranged may be identical between each of the respective electrodes. In other such embodiments the distance at which the electrodes are arranged may be identical between some of the respective electrodes. In yet other embodiments where more than two electrodes are provided, each distance at which two electrodes are arranged may be different from another distance at which two electrodes are arranged.

As an illustrative example the distance at which the electrodes are arranged, for instance a gap between two electrodes, may be in a range that corresponds to the length of a respective analyte molecule, such as a nucleic acid molecule. It is noted in this regard that for instance a linearised chromosome may have a length of up to 1.5 m (http://hypertextbook.com/facts/1998/StevenChen.shtml). As a further illustration, already Watson and Crick were able to determine the distance between the two strands of DNA as 2 nanometres. From their DNA model the vertical rise per base pair along the axis of a DNA molecule can be calculated to be 0.34 nm. Typical DNA molecules in human blood plasma have furthermore been reported to be of a length of 100 to 900 nm (http://cat.inist.fr/?aModele=afficheN&cpsidt=2324077). In some embodiments the distance at which the electrodes are arranged is of the same or a smaller length than the length of the analyte molecule. In such embodiments the analyte molecule is capable of spanning the respective gap. A respective distance, e.g. a gap, may for instance have a with selected in the range of about 0.5 nm to about 10 μm, such as a range of about 1 nm, or about 10 nm to about 200 nm, about 300 nm, about 500 nm, about 700 nm, about 800 nm or about 1 μm or 2 μm. As two illustrative examples, a distance of 30 nm may be selected, which would roughly correspond to a length of a linear nucleic acid of about 100 bp. (Such an estimate can be made based on the known helical pitch of ideal A, B and Z DNA for example. B DNA, for example, has a height of 0.34 nm per helical turn and base pair so that 10 base pairs (bp) bridge a distance of 3.4 nm). Alternatively, the distance with can be determined empirically for longer non linear nucleic acids; a distance of 200 nm, may roughly correspond to a length of a nucleic acid of about 2000 to 5000 bp.

As an illustrative example, a target nucleic acid molecule may be of for instance 100-500 nm, which is for example of a sufficient size of a nucleic acid molecule to includes exemplary genes. The capture molecule may in such an embodiment be immobilised in vicinity to the region in between the electrodes, or even within the respective region. In embodiments where this region in between the electrodes is defined by a small distance separating the electrodes, such as e.g. about 20-about 30 nm, the size of such a nucleic acid analyte molecule will allow the biological analyte molecule to bridge the respective distance between the electrodes (e.g. a gap). Thus, the present invention provides a method by which a single biological analyte molecule can be detected.

The method of the invention includes providing an immobilisation unit. A respective immobilisation unit may be of any material as long as an electrical measurement can be carried out. It may be desired to select the material of the immobilisation unit in order to immobilise a capture molecule thereon (see below). The surface of the immobilisation unit, or a part thereof, may also be altered, e.g. by means of a treatment carried out to alter characteristics thereof. Such a treatment may include various means, such as mechanical, thermal, electrical or chemical means. As an illustrative example, the surface properties of any hydrophobic surface can be rendered hydrophilic by coating with a hydrophilic polymer or by treatment with surfactants. Examples of a chemical surface treatment include, but are not limited to exposure to hexamethyldisilazane, trimethylchlorosilane, dimethyldichlorosilane, propyltrichlorosilane, tetraethoxysilane, glycidoxypropyltrimethoxy silane, 3-aminopropyltriethoxysilane, 2-(3,4-epoxy cyclohexyl)ethyltrimethoxysilane, 3-(2,3-epoxy propoxyl)propyltrimethoxysilane, polydimethylsiloxane (PDMS), γ-(3,4-epoxycyclohexyl)ethyltrimethoxysilane, poly(methyl methacrylate) or a polymethacrylate co-polymer, urethane, polyurethane, fluoro-polyacrylate, poly(methoxy polyethylene glycol methacrylate), poly(dimethyl acrylamide), poly[N-(2-hydroxypropyl)methacrylamide] (PHPMA), α-phosphorylcholine-o-(N,N-diethyldithiocarbamyl)undecyl oligoDMAAm-oligo-STblock co-oligomer (cf. e.g. Matsuda, T., et al., Biomaterials, (2003), 24, 4517-4527), poly(3,4-epoxy-1-butene), 3,4-epoxy-cyclohexylmethyl-methacrylate, 2,2-bis[4-(2,3-epoxy propoxy)phenyl]propane, 3,4-epoxy-cyclohexylmethyl-acrylate, (3′,4′-epoxycyclohexylmethyl)-3,4-epoxycyclohexyl carboxylate, di-(3,4-epoxycyclo-hexylmethyl)adipate, bisphenol A (2,2-bis-(p-(2,3-epoxy propoxy)phenyl)propane) or 2,3-epoxy-1-propanol.

In some embodiments the surface of the immobilisation unit may for instance be coated with an electroconductive polymer, such as polypyrrole (Wang, J., et al., Anal. Chem. (1999) 71, 18, 4095-4099; Wang, J., et al., Anal. Chim. Acta (1999) 402, 7-12), polythiophene, polyaniline, polyacetylene, poly(N-vinyl carbazole), or a copolymer such as a copolymer of pyrrole and thiophene or a copolymer of juglone and 5-hydroxy-3-thioacetic-1,4-naphthoquinone (Reisberg, S., et al., Anal. Chem. (2005) 77, 10, 3351-3356). In embodiments where the immobilisation unit is included in a surface of a carbon paste electrode, it may for example be modified with carboxyl groups by mixing stearic acid with the paste. The linking molecule ethylenediamine may for instance be immobilised on a respective electrode in order to facilitate the subsequent immobilisation of a capture molecule (see below).

The immobilisation unit is arranged within the sensing zone. The sensing zone is usually a region or aperture into which the analyte molecule is caused to be located. As two illustrative examples, the sensing zone may be a region or aperture to which the analyte molecule is caused to flow or into which the analyte molecule is disposed. In typical embodiments the sensing zone is defined by the zone in which an electric field of the pair of electrodes is effective. In some embodiments the immobilisation unit is arranged between two electrodes that are used to generate an electric field (see also below). In some of these embodiments the immobilisation unit is arranged in the gap of a detection electrode. In some embodiments the immobilisation unit is included on an electrode (e.g. a detection electrode). A respective detection electrode may for example be used for the detection of an electric signal in the method of the present invention (see below). As an example, a respective detection electrode may be used for the generation of an electric field. In some embodiments the immobilisation unit is conductively connected to an electrode.

In the method of the invention a nucleic acid capture molecule with an at least essentially uncharged backbone is immobilized on the detection electrode. The term “essentially uncharged” refers to the selected conditions of pH and ionic strength. Typically the method of the invention will be carried out at a pH in the range of about 5 to about 8 or to about 9, for instance at about neutral pH, i.e. about pH 7. Those skilled in the art will be aware that the pH will generally be above that at which C and A bases ionize (about 3) and below that at which G and T bases ionize (about 11). The nucleic acid backbone may also remain uncharged at pH's outside a respective condition and/or range.

Examples of a nucleic acid capture molecule with an at least essentially uncharged backbone include, but are not limited to, a PNA capture molecule, an alkylphosphonate nucleic acid capture molecule, an alkylphosphotriester nucleic acid capture molecule, an N3′→P5′-phosphoamidate nucleic acid capture molecule, a dialkylene sulfone nucleic acid capture molecule, a sulphonamide nucleic acid capture molecule, a sulfonate nucleic acid capture molecule, a sulfamate nucleic acid capture molecule, an alkylhydroxylamine nucleic acid capture molecule (Vasseur, J.-J., et al., J. Am. Chem. Soc. (1992) 114, 4006-4007), a methylene (alkylimino) nucleic acid capture molecule (Morvan, F., et al., J. Am. Chem. Soc. (1996) 118, 255-256), an amide nucleic acid capture molecule (De Mesmaeker, A., et al., Angew. Chem., Int. Ed. Engl. (1994) 33, 226-229), a formacetal nucleic acid capture molecule (Matteucci, M., Tetrahedron Lett. 1990, 31, 17, 2385-2388; Jones, R. J., et al., J. Org. Chem. (1993) 58, 2983-2991), a thioformacetal nucleic acid capture molecule (Jones, et al., 1993, supra) and a chimera of any of these. An introduction into the various backbone modifications available can be found in the review by Micklefield (Current Medical Chemistry (2001) 8, 1157-1179). As indicated above, a PNA molecule is a nucleic acid molecule in which the backbone is a pseudopeptide rather than a sugar. Accordingly, PNA generally has a charge neutral backbone, in contrast to DNA or RNA. Nevertheless, PNA is capable of hybridising at least complementary and substantially complementary nucleic acid strands, just as e.g. DNA or RNA (to which PNA is considered a structural mimic). PNA is furthermore generally nuclease/protease resistant. The term “PNA molecule” is understood in the art—and accordingly used in the context of the method of the invention—to include analogues and derivatives in which the backbone or bases are modified. As an illustrative example, the peptide backbone of a PNA molecule may include additional ester linkages to increase the flexibility of the molecule and thus facilitate hybridization to e.g. DNA or RNA. An overview on various forms of PNA has been given by Porcheddu & Giacomelli (Current Medicinal Chemistry (2005) 12, 2561-2599) and an overview on the use of PNAs as probes for nucleic acids, i.e. as nucleic acid capture molecules, has been given by Pellestor & Paulasova (European Journal of Human Genetics (2004) 12, 694-700).

Alkylphosphonate and alkylphosphotriester nucleic acid molecules can be viewed as a DNA or an RNA molecule, in which phosphate groups of the nucleic acid backbone are neutralized by exchanging the P—OH groups of the phosphate groups in the nucleic acid backbone to an alkyl and to an alkoxy group, respectively. Examples of an alkyl-phosphotriester nucleic acid are methylphosphotriester DNA and RNA (see e.g. Buck, H. M., Nucleosides, Nucleotides & Nucleic Acids (2004) 23, 12, 1833-1847; Buck, H. M., Nucleosides, Nucleotides & Nucleic Acids (2007) 26, 205-222). Methylphosphotriester RNA is capable of hybridizing to both DNA and RNA and has thus been used for genetic inhibition on the DNA and the RNA level (Buck, 2007, supra). In contrast thereto, methylphosphotriester DNA is so far only known to hybridize to DNA. Neutral and/or zwitterionic nucleic acid capture molecules containing bicyclo-deoxynucleosides and/or amino-bicyclo-deoxynucleosides as described by Meier et al. (Helvetica Chimica Acta (1999) 82, 1813-1828), are generally suitable for the method of the invention and can be tested in a respective method where desired.

The nucleic acid capture molecule may be immobilised according to any desired protocol. A general orientation on practical aspects of the technique as well as an indication on procedural steps that may be advisable to include or to utilize can for instance be found in the protocol of Strohsahl et al. (Nature Protocols (2007) 2, 9, 2105-2110). In some embodiments the nucleic acid capture molecule may be deposited on the surface of the immobilisation unit by any suitable protocol, e.g. contact printing such as pin printing or microstamping, photochemistry-based printing, laser writing, electrospray deposition, electro-printing or droplet dispensing (for an overview see Barbulovic-Nad, I., et al., Critical Reviews in Biotechnology (2006) 26, 237-259). The nucleic acid capture molecule used in a method according to the present invention may be of any suitable length. In some embodiments it has a length of about 7 to about 30 bp, for example a length of about 9 to about 25 bp, such as a length of about 10 to about 20 bp.

The nucleic acid capture molecule includes a nucleotide sequence that is at least partially complementary to at least a portion of a strand of the analyte molecule. As an illustrative example, a single-stranded nucleic acid molecule may be selected as the nucleic acid capture molecule. Such a single-stranded nucleic acid molecule may have a nucleic acid sequence that is at least partially complementary to at least a portion of a strand of the nucleic acid molecule that is the analyte molecule. The respective nucleotide sequence of the nucleic acid capture molecule may for example be 70, for example 80 or 85, including 100% complementary to another nucleic acid sequence. The higher the percentage to which the two sequences are complementary to each other (i.e. the lower the number of mismatches), the higher is typically the sensitivity of the method of the invention. In typical embodiments the respective nucleotide sequence is substantially complementary to at least a portion of the target nucleic acid molecule. “Substantially complementary” as used herein refers to the fact that a given nucleic acid sequence is at least 90, for instance 95, such as 100% complementary to another nucleic acid sequence. The term “complementary” or “complement” refers to two nucleotides that can form multiple favourable interactions with one another. Such favourable interactions are specific association between opposing or adjacent pairs of nucleic acid (including nucleic acid analogue) strands via matched bases, and include Watson-Crick base pairing. As an illustrative example, in two given nucleic acid molecules (e.g. DNA molecules) the base adenosine is complementary to thymine or uracil, while the base cytosine is complementary to guanine.

A nucleotide sequence is the complement of another nucleotide sequence if all of the nucleotides of the first sequence are complementary to all of the nucleotides of the second sequence. Accordingly, the respective nucleotide sequence will specifically hybridise to, or undergo duplex formation with, the respective portion of the target nucleic acid molecule under suitable hybridisation assay conditions, in particular of ionic strength and temperature.

In some embodiments the target nucleic acid molecule includes a pre-defined sequence. In some embodiments the target nucleic acid molecule furthermore includes at least one single-stranded region. In such embodiments it may be desirable to select a single-stranded region as the predefined sequence. In this case the nucleic acid capture molecule can directly form Watson-Crick base pairs with the target nucleic acid molecule, without the requirement of separating complementary strands of the nucleic acid analyte molecule. Where the nucleic acid molecule that is the analyte molecule, or a region thereon that includes e.g. a predefined sequence, is provided or suspected to be in double strand form, the respective nucleic acid duplex may be separated by any standard technique used in the art, for instance by increasing the temperature (e.g. 95° C., see also the Examples below). In embodiments where multiple sequences may be included in the target nucleic acid molecule, multiple respective capture molecules may be used, each of which being at least partially complementary to e.g. a selected portion of the target nucleic acid molecule (see also below).

As explained above, the nucleic acid capture molecule is often a single-stranded nucleic acid molecule. By hybridisation of the two nucleic acid molecules, i.e. the nucleic acid capture molecule and the target nucleic acid molecule, a complex is formed. It is understood that for the quantification of such a nucleic acid molecule a plurality of the respective capture molecules is usually required. In a suitable concentration range of the target nucleic acid molecule, where the method of the invention can be used to quantify a respective target nucleic acid molecule, generally an excess of nucleic acid capture molecules in comparison to target nucleic acid molecule is required. As a result, one or more single-stranded nucleic acid capture molecules, which do not form a complex with an analyte molecule, may remain. Depending on the electroconductive wire(s) formed (see below), the presence of such a nucleic acid capture molecule may interfere with the detection of the method of the present invention. In particular where the nucleic acid capture molecule is a single-stranded DNA molecule or a single-stranded RNA molecule, such a remaining nucleic acid molecule may be removed from the immobilisation unit.

For detecting an analyte molecule, the electrical characteristic of a region in the sensing zone, e.g. the region in between the electrode arrangement, must be influenced by the electrical conductivity of the metal wire associated with the nucleic acid capture molecule/target nucleic acid molecule complex. For this it is sufficient that that the immobilisation unit, or at least the surface or a part of the surface thereof, is either located in vicinity to the electrodes of the electron pair or in electrical communication, e.g. electrically connected thereto. In these embodiments, the (immobilised) complex of the nucleic acid capture molecule molecule (in particular a nucleic acid molecule with a larger size, e.g. of several thousands or more base pairs) with the electrically conducting wire may, for example, swing by Brownian motion with any flexible part (or parts) thereof into the distance in between the electrodes. Alternatively, the electrical interaction between the wire and an electrical field applied at the electrodes can alone be sufficient to influence the electrical characteristics in the gap in between the electrodes in a detectable manner. In other embodiments the respective immobilisation surface of the immobilisation unit is arranged within the respective region defined by the distance between the (or some of the) electrodes. In some further embodiments the surface of the immobilisation unit is included on an electrode (e.g. a detection electrode). A respective detection electrode may for example be used for the detection of an electric signal in the method of the present invention (see below). As an illustrative example, a respective detection electrode may be used for the generation of an electric field. In some embodiments the surface is conductively connected to an electrode.

As already indicated above, in some embodiments the immobilisation unit is included in or on (e.g. included in the surface of) or conductively connected to an electrode. As an illustrative example, the immobilisation unit may be the surface of a detection electrode or included in the surface of a detection electrode.

In some embodiments the immobilisation unit is located on a semiconductor based transistor or conductively connected thereto. As an example, the surface of the immobilisation unit may be or be included in the surface of a gate electrode of a field effect transistor (FET; see e.g. Ingebrandt, S., et al., Biosensors & Bioelectronics (2007) 22, 2834-2840 or Maki, W. C., et al., Biosensors and Bioelectronics (2007), doi:10.1016/j .bios.2007.08.017, for an overview see Schöning & Poghossian, 2002, supra). In some embodiments the immobilisation unit is conductively connected to the gate electrode of a field effect transistor (FET) as for instance disclosed in US patent application 2006/0029994. The immobilisation unit may also be or included in at least a part of a floating gate electrode of a field effect transistor as described by Barbaro et al. (IEEE Transactions on electron devices [2006], 53, 1, 158-166, see also below). In some embodiments the immobilisation unit is electrically conductive. In other embodiments the immobilisation unit is an electrical insulator, but becomes electrically conductive once a metal wire has been formed thereon in the method of the present invention (see below). In this regard, the terms “electroconductive”, “electrically conducting” and “electrically conductive”, are used interchangeably herein, and refer to the capability to carry current or otherwise transmit electricity, as opposed to an insulator, the latter having a high electrical resistivity and low electrical conductivity.

The method of the invention further includes immobilising the nucleic acid capture molecule on the immobilisation unit, generally on a surface or a part of a surface of an immobilisation unit. The respective surface (or surface part) of the immobilisation unit is arranged within the sensing zone. In some embodiments at least a part of the respective surface of the immobilisation unit is arranged in a zone where an electric field of the pair of electrodes is effective. In some embodiments upon immobilisation of the capture molecule at least a part thereof is included in the region defined by the distance between the (or some of the) electrodes.

The nucleic acid capture molecule may be immobilized on the immobilisation unit at any stage during the present method of the invention. As two examples, it may be immobilized at the beginning of the method or before adding an activation agent (see below). In typical embodiments it is immobilised before performing an electrochemical measurement (see below). The nucleic acid capture molecule may be immobilized by any means. It may be immobilized on the entire surface, or a selected portion of the surface of the detection electrode. In some embodiments the PNA capture molecule is provided first and thereafter immobilized onto the surface of the detection electrode. An illustrative example is the mechanical spotting of the PNA capture molecule onto the surface of the electrode. This spotting may be carried out manually, e.g. by means of a pipette, or automatically, e.g. by means of a micro robot. As an illustrative example, the polypeptide backbone of the PNA capture molecule may be covalently linked to a gold detection electrode via a thioether bond.

In some embodiments the nucleic acid capture molecule is immobilised on the immobilisation unit via a covalent bond. In some embodiments a linking molecule may be used to attach the capture molecule to the immobilisation unit (see also above). Any molecule with a reactive moiety that is capable of undergoing a reaction with a corresponding moiety of an analyte molecule may be used. As an illustrative example, a linking molecule may be an aliphatic compound with a backbone of about 4 to about 50 carbon atoms, of which some may be exchanged by N, O, Si or S atoms, and a reactive functional group. Examples of reactive functional groups include, but are not limited to, aldehydes, carboxylic acids, esters, imido esters, anhydrides, acyl nitriles, acyl halides, acyl azides, isocyanates, sulphonate esters, sulfonyl halides, or aryl halides, which may for example react with an amino group of a capture molecule, or alkyl sulphonates, aryl halides, acrylamides, maleimides, haloacetamides or aziridines, which may for example react with a thio group of a capture molecule or a carboxylic acid, an anhydride, an isocyanate, a phosphoramidite, a halotriazine, an acyl halide, an acyl nitrile, an alkyl halide, an alkyl sulphonate or a maleimide, which may for example react with a hydroxy group of a capture molecule. In embodiments where the immobilisation unit includes or consists of silicon, a respective surface can also be functionalised via a cyclic voltametric reduction process using a diazonium compound and glutaraldehyde, so that an aldehyde group is provided for the covalent coupling of a nucleic acid molecule (Shabani, A., et al., Talanta (2007) 70, 615-623). Where the immobilisation unit includes or consists of gold, a bifunctional linker molecule that includes a thiol group and a carboxy group, separated by a spacer, may be coupled to the respective surface as described by Nakamura et al. (Colloids and Surfaces A (2006) 284/285, 495-498).

Any of the above examples of a capture molecule may also serve as a linker for the immobilisation of another capture molecule. This may for example be desired to obtain a capture molecule that has a chosen degree of specificity for a selected analyte molecule. Avidin or streptavidin may for instance be employed to immobilise a biotinylated nucleic acid, or a biotin containing monolayer of gold may be employed (Shumaker-Parry, J. S., et al., Anal. Chem. (2004) 76, 918). As another illustrative example, the capture molecule may be a metal ion bound by a respective metal chelator (see above). A capture molecule that is capable of forming a complex with a desired analyte molecule may then be equipped with an affinity tag for such a metal ion by means of genetic engineering. Upon contacting the ion, which is immobilised on the immobilisation unit via the respective metal chelator with such a capture molecule, the capture molecule is immobilised on the immobilisation unit. As yet another illustrative example, a nucleic acid capture molecule may be locally deposited, e.g. by scanning electrochemical microscopy, for instance via pyrrole-oligonucleotide patterns (e.g. Fortin, E., et al., Electroanalysis (2005) 17, 495). It is understood that in other embodiments a nucleic acid capture molecule may be directly synthesized on the immobilisation unit, for example using photoactivation and deactivation. In some embodiments the nucleic acid capture molecule may also be included in a nucleic acid-protein conjugate, such as a DNA-strepavidin conjugate (Niemeyer, C. M., Biochemical Society Transactions (2004) 32, 1, 51-53). The protein portion of such a conjugate may be selected for its capability of binding to a desired surface. In such embodiments, the additional further capture molecule may be seen as being included in the nucleic acid capture molecule.

Where desired an organic coating, e.g. a monolayer, may be formed on the immobilisation unit prior to immobilising the nucleic acid capture molecule thereon. As an illustrative example, a plasma-polymerised film (e.g. from hexamethyldisilazane) may be deposited on the surface of the immobilisation unit, and a nucleic acid molecule may be deposited by glutaraldehyde coupling, possibly following acrylamide grafting and poly-ethyleneimine treatment (Chen, K.-S., Materials, Science & Engineering C (2007) 27, 716-724). In embodiments where the immobilisation unit includes or consists of silicon, a phtalimide monolayer may be formed thereon, e.g. starting from vinylphthalimide, which has been shown to include the formation of covalent Si—C bonds (Ara, M., et al., Surface Science (2007) doi: 10.1016/j.susc. 2007.04.213). Such a monolayer provides amino groups for a subsequent immobilisation of nucleic acid molecules (ibid.). As an alternative, in such embodiments a layer of (3-mercaptopropyl)trimethoxysilane may be formed on the surface (Lenigk, R., et al., Langmuir (2001) 17, 2497-2501). A 5′-thiol-terminated nucleic acid capture molecule may be immobilised on such a pretreated surface.

In some embodiments the immobilisation unit can serve as a further electrode itself. The same methods of activating the surface of any other immobilisation unit may be applied to such an electrode prior to immobilizing the nucleic acid capture molecule thereon, for instance in order to facilitate the attachment reaction (see also above). Where a glass electrode is used, it may for example be modified with aminophenyl or aminopropyl silanes. 5′-succinylated nucleic acid capture molecules may be immobilised thereon by carbodiimide-mediated coupling. In some embodiments the electrode surface may for instance be coated with an electroconductive polymer, such as polypyrrole (Wang, J., et al., Anal. Chem. (1999) 71, 18, 4095-4099; Wang, J., et al., Anal. Chim. Acta (1999) 402, 7-12), polythiophene, polyaniline, polyacetylene, poly(N-vinyl carbazole), or a copolymer such as a copolymer of pyrrole and thiophene or a copolymer of juglone and 5-hydroxy-3-thioacetic-1,4-naphthoquinone (Reisberg, S., et al., Anal. Chem. (2005) 77, 10, 3351-3356). In embodiments where a carbon paste electrode is used, it may for example be modified with carboxyl groups by mixing stearic acid with the paste. A nucleic acid capture molecule may be immobilised on a respective electrode by means of linking molecule ethylenediamine.

As a further illustrative example, a linking moiety such as an affinity tag may be used to immobilize the nucleic acid capture molecule. Examples of an affinity tag include, but are not limited to biotin, dinitrophenol or digoxigenin, oligohistidine, polyhistidine, an immunoglobulin domain, maltose-binding protein, glutathione-5-transferase (GST), calmodulin binding peptide (CBP), FLAG′-peptide, the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the hemagglutinin (HA) epitope of the sequence Tyr-Pro-Tyr-Asp-Val-Pro-Asp-Tyr-Ala, the “myc” epitope of the transcription factor c-myc of the sequence Glu-Gln-Lys-Leu-Ile-Ser-Glu-Glu-Asp-Leu, or an oligonucleotide tag the sequence of which typically differs from the sequence of the target nucleic acid molecule to which a portion of the nucleic acid capture molecule is at least partially complementary. These two nucleotide sequences may differ to such an extent that the sequence of the nucleotide tag is not capable of hybridising to the sequence of any portion of the target nucleic acid molecule. Such an oligonucleotide tag may for instance be used to hybridise to an immobilized oligonucleotide with a complementary sequence. A further example of a linking moiety is an antibody. In respective embodiments an antibody-nucleic acid conjugate may be used as the nucleic acid capture molecule.

Avidin or streptavidin may for instance be employed to immobilize a biotinylated nucleic acid, or a biotin containing monolayer of gold may be employed (Shumaker-Parry, J. S., et al., Anal. Chem. (2004) 76, 918). As yet another illustrative example, the nucleic acid capture molecule may be locally deposited, e.g. by scanning electrochemical microscopy, for instance via pyrrole-oligonucleotide patterns (e.g. Fortin, E., et al., Electro-analysis (2005) 17, 495). In other embodiments the nucleic acid capture molecule (e.g. a PNA capture molecule) may be directly synthesized on the detection electrode, for example using photoactivation and deactivation.

If desired, more than one capture molecule may be immobilised on the immobilisation unit. This may for instance be desired in order to broadly screen for the presence of any of a group of selected nucleic acid sequences. This may also be desired to allow for the simultaneous or consecutive detection of different target nucleic acids such as two or more genomic DNAs, each of them having binding specificity for one particular type of capture molecule. In some embodiments similar nucleic acid sequences, e.g. a number of nucleic acid sequences that are partially or substantially complementary to a selected nucleic acid analyte molecule, may be immobilised in order to enhance the likelihood of detecting the respective nucleic acid analyte molecule. Two or more capture molecules may also be desired in order to be able to detect two or more analyte molecules. In some embodiments of the method of the invention the presence of one analyte molecule may also provide confirmation of the presence of another analyte molecule.

After immobilizing the nucleic acid capture molecule on the detection electrode, any remaining nucleic acid capture molecule, or molecules, that were not immobilized may be removed from the detection electrode. Removing an unbound nucleic acid capture molecule may be desired to avoid subsequent hybridisation of such capture molecule with the target nucleic acid molecule, which might reduce the sensitivity of the present method. Removing an unbound nucleic acid capture molecule may also be desired to avoid a non-specific binding of such capture molecule to any matter present in a sample used, which might for instance alter the conductivity of such matter (e.g., reducible metal cations), which might interfere with the results of the electrochemical measurement (see also below). An unbound capture molecule may for instance be removed by exchanging the medium, e.g. a solution that contacts the detection electrode.

Where desired, a blocking agent may be immobilised on the detection electrode. This blocking agent may serve in reducing or preventing non-specific binding of matter included in the solution expected to include the target nucleic acid molecule. It may also serve in reducing or preventing non-specific binding of any other matter, such as a molecule or solution that is further added to the detection electrode when carrying out the method of the invention.

The blocking agent may be added together with the nucleic acid capture molecule or subsequently thereto. Any agent that can be immobilized on the electrode and that is able to prevent (or at least to significantly reduce) the non-specific interaction between molecules, the detection of which is undesired, and the nucleic acid capture molecule is suitable for that purpose, as long as the specific interaction between the nucleic acid capture molecule and the target nucleic acid molecule is not prevented. Examples of such agents are thiol molecules, disulfides, thiophene derivatives, and polythiophene derivatives. An illustrative example of a useful class of blocking reagents include thiol molecules with about 8 to about 25 main chain atoms such as 16-mercaptohexadecanoic acid, 12-mercaptododecanoic, 11-mercaptodecanoic acid or 10-mercaptodecanoic acid.

The term “derivative” as used herein thus refers to a compound which differs from another compound of similar structure by the replacement or substitution of one moiety by another. Respective moieties include, but are not limited to atoms, radicals or functional groups. For example, a hydrogen atom of a compound may be substituted by alkyl, carbonyl, acyl, hydroxyl, or amino functions to produce a derivative of that compound. Respective moieties include for instance also a protective group that may be removed under the selected reaction conditions.

The method of the present invention further includes contacting the immobilisation unit with a solution suspected to include the analyte molecule (see also above). The immobilisation unit may for example be immersed in a solution, to which the solution suspected to include the analyte acid molecule is added. In some embodiments both such solutions are aqueous solutions. In one embodiment the entire method is carried out in an aqueous solution. The method further includes allowing the target nucleic acid molecule to form a complex with the nucleic acid capture molecule on the immobilisation unit. This occurs by allowing the target nucleic acid molecule to hybridise to the nucleic acid capture molecule on the immobilisation unit. If the solution contains a plurality of different target nucleic acid molecules to be detected, the conditions may be chosen so that the target nucleic acid molecules can either bind simultaneously or consecutively to their respective capture molecules.

As explained above, the nucleic acid capture molecule is typically a single-stranded nucleic acid molecule. By hybridisation of the two nucleic acid molecules, i.e. the capture molecule and the analyte molecule, a complex is formed. It is understood that at least for the quantification of such a nucleic acid molecule a plurality of the respective capture molecules is usually required. In a suitable concentration range of the analyte molecule, where the method of the invention can be used to quantify a respective analyte molecule, generally an excess of capture molecules in comparison to analyte molecule is required. As a result, one or more single-stranded nucleic acid capture molecules, which do not form a complex with an analyte molecule, may remain. Depending on the activation agent, the polymer and the metal salt used (see below), the presence of such a nucleic acid capture molecule may interfere with the detection of the method of the present invention. In particular where the nucleic acid capture molecule is a single-stranded DNA molecule or a single-stranded RNA molecule, such a remaining nucleic acid molecule may be removed from the immobilisation unit.

In such embodiments the immobilisation unit may be contacted with at least one enzyme with nuclease activity, in order to remove any nucleic acid capture molecule that has not hybridised to an analyte molecule. It may be desired to reduce or block nuclease activity that is directed against double-strands of nucleic acids in order to avoid a reduction of detection signal, caused by the degradation of complexes of capture molecule and analyte. In some embodiments an enzyme may be selected that selectively degrades single-stranded nucleic acids. Examples of such enzymes include, but are not limited to, mung bean nuclease, nuclease P1 (e.g. from fungi), nuclease S1 (e.g. from fungi), CEL I nuclease (e.g. from plants), recJ exonuclease (e.g. from E. coli), and a DNA polymerase that is capable of degrading single-stranded DNA due to its 5′->3′ exonuclease activity and a DNA polymerase that is capable of degrading single-stranded DNA due to its 3′->5′ exonuclease activity.

The method of the present invention further includes adding an activation agent. The activation agent has an electrostatic net charge that is complementary to the electrostatic net charge of the target nucleic acid molecule. As explained above a DNA or RNA molecule with an unmodified backbone possesses negative charges on the latter and thus has an overall negative net charge. The activation agent may for example have an affinity to phosphate and/or phosphonate, which may allow the activation agent to associate to the target nucleic acid molecule, for example at the backbone thereof.

The activation agent can typically interact chemically with the target nucleic acid molecule. A chemical interaction, which is generally an intermolecular interaction and generally creates an attractive force, may include the formation of non-covalent or covalent bonds, including van der Waals force, polar interaction (such as dipole-dipole interaction or ionic interaction), complex formation, etc. The activation agent may for example include or consist of a metal or a metalloid such as a metal oxide or a metal hydroxide. The metal or metalloid may have an affinity for the analyte molecule. The activation agent may in some embodiments be able to form a complex with the target nucleic acid molecule via a coordinative bond. A respective coordinative bond may in some embodiments be formed between one or more metal atoms of a metal or metal oxide included in or associated to the activation agent. A respective coordinative bond may for example be formed by a reaction of the activation agent with the target nucleic acid molecule.

In some embodiments the metal or metalloid is capable of forming a complex with the analyte molecule via negative charges, which are present on the surface of the respective nucleic acid molecule (see above). As an illustrative example, a metal oxide or a metal hydroxide may have an affinity to phosphate and/or phosphonate. Such a metal oxide is therefore able to associate with nucleic acid molecules that contain phosphate or phosphonate groups such as DNA and RNA, which contain a phosphate backbone. An activation agent that contains or consists of a respective metal oxide associate to the backbone of a nucleic acid molecule. Nucleic acid alkyl or aryl phosphonates analogues may for example be included in DNA mimics, such as analogues of peptide nucleic acids.

Examples of a suitable metal oxide with an affinity to phosphate and/or phosphonate include, but are not limited to indium tin oxide, titanium oxide, tantalum oxide, copper oxide and zinc oxide. Examples of a suitable metal hydroxide with an affinity to phosphate and/or phosphonate include, but are not limited to aluminiumhydroxide, titanium hydroxide, copper hydroxide and gold hydroxide Au(OH)₃. Phosphonic acids have for example been shown to adsorb to many metal oxides such as copper oxide, silver oxide, titanium oxide, aluminium oxide, zirconium oxide or ferric oxide and to form monolayers thereon (Folkers, J. P. et al. Langmuir [1995] 11, 813-824). The adsorption and self-assembly of a monolayer of octadecylphosphoric acid on a surface tantalum oxide Ta₂O₅ has for instance been reported, which has been suggested to be based on coordinative bonds (Brovelli, D., et al., Langmuir [1999] 15, 4324-4327; Textor, M., et al., Langmuir [2000] 16, 3257-3271). It has been speculated that an oxide ion in the Ta₂O₅ surface is being replaced by phosphate via a protonation of the oxide (Textor et al., 2000, supra), as schematically summarised by the scheme:

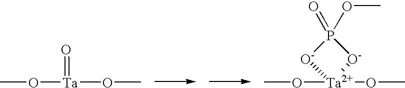

As a further example, DNA has been found to associate to indium tin oxide surfaces (abstract Q1.00105 of 2006 March meeting of the American Physical Society).

As noted above, the activation agent may include or consist of a metal atom, such as a transition metal atom, or a metalloid atom such as a silicon atom. It may for example be a transition metal compound such as a zirconium compound or a niobium compound. Zirconium ions are for example known to coordinatively achieve the association of a phosphate- to a carboxylate moiety or of different phosphate moieties (Mazur, M., et al. Langmuir [2005] 21, 8802-8808; Lee, H., et al., J. Phys. Chem. [1998] 92, 2597-2601). It is known that Zr⁴⁺ ions coordinate more than one phosphate or phosphonate molecule and that phosphate or phosphonate moieties bind to more than one metal ion (see e.g. Bujoli, B. et al., Progress in Solid State Chemistry (2006) 34, 257-266 or Lane, S. M., et al., Colloids and Surfaces B: Biointerfaces (2007) 58, 34-38).

An illustrative example of a suitable zirconium compound is zirconyl chloride. Previously it has been shown that oligonucleotides can be immobilised on a glass surface that was coated with indium tin oxide (Zeng, J., & Krull, U. J., Chimica Oggi [2003] 21, 10/11, 48-52), when zirconyl chloride octahydrate and either sodium sulphate or 4-formylphenyl phosphate were employed. The formation of zirconium sulphate using sodium sulphate, and the formation of aldehyde moieties on the immobilisation unit using formylphenyl phosphate apparently resulted in the immobilisation of oligonucleotides. The present inventors have previously shown that the bond formed between zirconium ions and the phosphate backbone of nucleic acids is stable enough to allow for the immobilisation of indium tin oxide nanoparticles thereon (Fan, Y., et al., Angew. Chem. Int. Ed. (2007) 46, 2051-2054). Further examples of compounds suitable as an activation agent include, but are not limited to, silica (SiO₂), titania (TiO₂) or niobium oxide (Nb₂O₅).

For illustration purposes the activation agent may be thought of as defining an anchor allowing the polymer (see below) to be immobilised to the complex formed between the nucleic acid capture molecule and the target nucleic acid molecule. The activation agent may be added in any quantity that does not interfere with the stability of the complex of target nucleic acid molecule and nucleic, acid capture molecule, and which does not impede the association of the polymer used (see below). In typical embodiments an excess of activation agent with respect to the number of nucleic acid capture molecules immobilised on the immobilisation unit is added. As an example, the amount of activation agent added may be of an excess, e.g. a molar excess, such as about 5-fold, 10-fold, 25-fold, 50-fold, 100-fold or several hundred times the number of nucleic acid capture molecules on the immobilisation unit. For this purpose the number of nucleic acid capture molecules that have been immobilised on the immobilisation unit may be calculated or analytically determined. Once the activation agent has been allowed to associate to the target nucleic acid/nucleic acid capture molecule complex, any non-associated activation agent may be removed, for example by means of rinsing or washing.

Those skilled in the art will appreciate that the method of the present invention is based on the addition of matter to the target nucleic acid/capture nucleic acid complex that is able to bind to an analyte molecule, i.e. a target nucleic acid molecule, in itself, rather than relying on affinity tags that need to be linked to matter added or to the target nucleic acid molecule. The use of affinity tags (see e.g. Luo, X., et al., Electroanalysis [2006], 18, 319-326; or Rosi, N. L. Chem. Rev. [2005] 105, 1547-1562) bears in particular the disadvantages of introducing complexity into a detection method and restructuring the surface or geometry of the respective matter.

The method of the present invention further includes adding a water soluble polymer, including any acceptable salt thereof (e.g., a sodium salt, ammonium or a potassium salt, to name only a few illustrative examples). This polymer has an electrostatic net charge that is complementary to the electrostatic net charge of the activation agent. If the activation agent is of positive net charge, associated to a negatively charged target nucleic acid molecule (which is in a complex with the nucleic acid capture molecule), the water soluble polymer will be selected to be of a negative net charge. As an illustration, a respective polymer may include at least one polymer strand. Examples of suitable water soluble polymers include, but are not limited to, a sulfated and sulfonated polymer such as a polystyrene derivative such as polystyrene sulfonate, poly(vinylsulfate), poly(propenesulfate), poly(butenesulfate), poly(pentenesulfate), poly(hexenesulfate), poly(heptenesulfate), poly(octenesulfate), poly(nonenesulfate), poly(decenesulfate), poly(undecenesulfate), poly(dodecenesulfate)Poly(vinylsulfonate), poly(propenesulfonate), poly(butenesulfonate), poly(pentenesulfonate), poly(hexenesulfonate), poly(heptenesulfonate), poly(octenesulfonate), poly(nonenesulfonate), poly(decenesulfonate), poly(undecenesulfonate), or poly(dodecenesulfonate) which are described in U.S. Pat. No. 6,290,946 for example. Other suitable examples of water soluble polymers include polymers with phosphate groups such as Poly(vinylphosphate), poly(propenephosphate), poly(butenephosphate), poly(pentenephosphate), poly(hexenephosphate), poly(heptenephosphate), poly(octenephosphate), poly(nonenephosphate), poly(decenephosphate), poly(undecenephosphate), poly(dodecenephosphate)Poly(vinylphosphonate), polytpropenephosphonate), poly(butenephosphonate), poly(pentenephosphonate), poly(hexenephosphonate), poly(heptenephosphonate), poly(octenephosphonate), poly(nonenephosphonate), poly(decenephosphonate), poly(undecenephosphonate), poly(dodecenephosphonate), to name only a few examples (see also U.S. Pat. No. 6,290,946). Further illustrative examples of suitable polymers include, but are not limited to an ethylene/maleic anhydride copolymer, a styrene/maleic anhydride copolymer, polyacrylic acid, poly(propylacrylic acid), poly(2-acrylamido-2-methylpropanesulfonic acid), poly(styrene-co-p-vinylbenzoic acid), poly(2-methoxyaniline-5-sulfonic acid), poly(m-xylene-5-carboxy-m-xylene) (Chemical Abstracts No 174715-05-0), poly(oxyethylene)diglycolic acid, starch glycolate, anionic polygalacturonic acid derivatives, carboxymethylcellulose, carboxymethyl inulin, an algin, an alginate, a hyaluronan, pectin, heparin, a chondroitin sulfate, an anionic carageenan derivative, a xantham gum, polyglutamate, and fragments, derivatives and salts thereof.

Further anionic polyalcohol derivatives may be used, or fragments thereof. Anionic moieties with which the polyalcohol can be derivatized include, for instance, carboxylate, phosphate or sulfate groups. An example of an anionic polymer is an anionic polysaccharide derivative, or a fragment thereof. The water soluble polymer may include or consist of a single macromolecule or a plurality thereof. It may include a single molecular species, such as a single type of polymer, or two or more different molecular species, such as a mixture of two or more types of polymers. The water soluble polymer may be biodegradable or non-biodegradable. Examples of cationic polymers include poly-L-lysine and other polymers of basic amino acids, a polyvinylamine, poly(4-vinyl pyridine), and poly(1-vinyl imidazole).

The water soluble polymer used in the present invention can have any molecular weight, as long as it is sufficiently soluble under the conditions used for the detection of the target nucleic acid and does not interfere with the detection. In some embodiments, water soluble polymer may have a molecular weight of about 1000 Da (1 kD) to about 2 million Daltons. In other embodiments, the molecular weight of the water soluble polymer may be about 5 kDa, about 10 kDa, about 50 kDa, about 70 kDa, about 100 kDa, about 500 kDa, about 750 kDa, about 1 million Da, about 1.25 million Da, about 1.5 million Da, or about 1.75 million Da.

In the method of the invention a metal salt is furthermore added. In typical embodiments the metal salt is added after the water soluble polymer has been added. The metal ions of the metal salt have an electrostatic net charge that is complementary to the electrostatic net charge of the water soluble polymer. In embodiments where the respective polymer has a negative net charge, the metal are therefore of positive charge. Any counter-ion (in the foregoing example of negative charge) may be used, whether organic or inorganic, as long as the metal ions are able to dissociate therefrom and to associate to the polymer. The metal ions of the metal salt are accordingly allowed to associate to the water soluble polymer. As an illustrative example in this regard, it has previously been demonstrated that the binding of the charged water soluble polymer poly(styrenesulfontate) to Cu²⁺ ions is strong enough to prevent the removal of associated Cu²⁺ ions during ultrafiltration (Moreno-Villoslada, I., et al., Macromol. Rapid Commun. (2001) 22, 14, 1191-1193).

As a result a plurality of metal ions binds to the polymer and covers the surface thereof. As a figurative illustration, the polymer may in some embodiments be thought of as including or consisting of one or more polymer strands (see also above). A plurality of the metal ions may then be thought of as binding thereto and covering the respective polymer strand(s), for example forming a structure resembling a string of pearls (e.g. a pearl necklet). It is understood that the metal ions may not necessarily form a simple array on the polymer and that any real arrangement or structure formed may significantly differ from this figurative illustration.

The metal salt may be added in any quantity that does not interfere with the stability of the target nucleic acid molecule/nucleic acid capture molecule complex, which does not disrupt, or if desired not impair, the association of the polymer thereto. In typical embodiments an excess of metal salt with respect to the number of nucleic acid capture molecules immobilised on the immobilisation unit is added. As an example, the amount of activation agent added may be of an excess, e.g. a molar excess, such as about 5-fold, 10-fold, 25-fold, 50-fold, 100-fold or several hundred times the number of nucleic acid capture molecules on the immobilisation unit. As noted above, the number of nucleic acid capture molecules on the immobilisation unit may be estimated, calculated or analytically determined in order to provide a desired ratio of metal salt molecules. Once the metal salt has been allowed to associate to the water soluble polymer that is associated to the target nucleic acid/nucleic acid capture molecule complex, any non-associated metal salt ions may be removed, for example by means of rinsing or washing.

The metal salt used in the method of the invention is capable of acting as an oxidant. Oxidation is a reaction in which an atom loses electrons. Accordingly, an oxidant, also termed oxidizing agent or oxidiser, is capable of oxidizing another substance by accepting electrons. In contrast thereto, a reducing agent is capable of furnishing electrons and thereby to reduce another substance. In some embodiments the metal ions have a standard electrode potential in the range from about +1 V to about 0 V, such as in the range from about +0.5 V to about 0 V. Examples of metal salts suitable for the method of the invention include, but are not limited to, a molybdenum salt, a copper salt, a germanium salt, a tin salt, a rhenium salt, an antimony salt, a platinum salt (e.g. Pt²⁺), a palladium salt (e.g. Pd²⁺, Pd⁴⁺), a silver salt (e.g. Ag⁺), a nickel salt, a cobalt salt (e.g. Co³⁺), an iron salt (e.g. Fe²⁺), a bismuth salt (e.g. Bi²⁺) and a mercury salt (e.g. Hg²⁺).

The method of the invention further includes adding a reducing agent. In typical embodiments the reducing agent is added after the metal salt has been added. The reducing agent is allowed to reduce the metal ions of the metal salt to the corresponding metal. As three illustrative examples, silver, copper or iron ions are accordingly allowed to be reduced to elementary silver, elementary copper or elementary ion. Any reducing agent may be used that is capable of reducing the metal salt and that does not abrogate a selected detection method. It may be desired to select a mild reducing agent in order to prevent undesired oxidation of other matter present, which may affect the electrical characteristic determined in the method of the invention. This may give rise to false signals, thereby reducing the sensitivity of the method. In some embodiments the reducing agent has a standard electrode potential in the range from about 0 V to about −1 V, such as in the range from about 0 V to about −0.5 V. Examples of a suitable reducing agent include, but are not limited to, ascorbic acid (vitamin C), oxalic acid, acetyl cysteine, thioglycolic acid, a thiosulphate (e.g. sodium thiosulphate), thiourea, dithiothreitol, a hydrosulphite, a bisulphite, a hydroquinone such as tertiary butyl hydroquinone (TBHQ), a borane, glucose, polyphenol compounds (for example, caffic acid, gallic acid, chlorgenic acid, sinapic acid, protocatechuic acid, 3-hydroxy, 5-hydroxy, 7-hydrohydroxyflavones, 3′,4′dihydroxyflavone, kaempferol, quercetin or luteolin, see Filipiak, M. Analytical Sciences 2001, Vol. 17 Supplement i1667 to 1671) and poly(vinyl pyrrolidone). In this regard the end groups of poly(vinyl pyrrolidone) have recently been shown to be fit for use as a mild reducing agent (Xiong, Y., et al., Langmuir (2006) 22, 8563-8570). An illustrative example of a borane is dimethylamine borane, the use of which in the presence of lactic acid and sodium citrate has been previously described in the reduction of palladium acetate to a palladium wire (Richter et al., 2001, supra).

As an illustrative example of a suitable reducing agent, ascorbic acid is a carboxylic acid with five hydroxy groups that intramolecularly forms a furan ring. Oxidation of ascorbic acid is known to be energetically favourable, so that ascorbic acid has reductive, and thus also antioxidant properties. Oxidation of (i.e. removal of electrons from) ascorbic acid is known to first yield monodehydroascorbate and—upon further oxidation—dehydroascorbate. Reduction by ascorbic acid has for instant been found to be an excellent agent for removal of oxidants generated during disinfection, where it is able to reduce oxidizing chlorine compounds (Urbansky, E. T., et al., Journal of Environmental Monitoring (2000) 2, 253-256). Its reductive properties are also the basis of an optical detection method of determining the presence and/or concentration of ascorbic acid via reduction of toluidine blue, which is decolourized by reduction (Safavi, A & Fotouhi, L., Talanta (1994) 41, 8, 1225-1228). The ability of ascorbic acid to reduce CuNO₃, associated to DNA, to elementary copper thereby forming a copper nanowire has previously been demonstrated (Monson & Woolley, 2003, supra). The Cu⁰ atoms act as a nucleation site for further metallization (ibid.), thus contributing to wire formation. The method of the present invention inter alia avoids the downside observed by Monson & Woolley that no wire formation occurred at certain DNA segments (ibid.).

As explained above, the metal ions, which are associated to the water soluble polymer, are reduced to the corresponding elementary metal. Accordingly, by reducing the metal ions of the metal salt, the reducing agent induces the formation of a metal wire. The length of the respective wire is only limited by the length of the polymer used and may be in the range from about a few nanometers to several millimetres or more. As an illustrative example, it may be of a range of about 100 nm to about 500 μm or to about 100 μm. A respective wire may for instance have a diameter of about 0.1 nm to about 10 μm, such as about 1 nm to about 1 μm, or about 10 nm to about 500 nm.

The method of the present invention further includes determining the presence of the analyte molecule based on an electrical characteristic of a region in the sensing zone. As an example, the immobilisation unit may be exposed to an electric field. The electric field may be generated by any means. It may in some embodiments be generated between two or more electrodes, such as in a two-, three- or four-electrode cell. Respective electrodes may be of any dimension, as long as an electric field can be generated that is sufficient to induce an electric signal caused by the electroconductive nanoparticle (see below). As already indicated above, in some embodiments the immobilisation unit may for example be part of the immobilisation unit of a respective electrode. In other embodiments it may be located in the gap between respective electrode.

In yet other embodiments the electric field is generated by a field effect transistor (FET), more specifically by two electrodes a field effect transistor, termed the ‘source’ and the ‘drain’. Field effect transistors are unipolar transistors in that only one type of charge, such as electrons, generates a current. A FET can be used to switch, to enhance or to deplete a current. In a FET current flows along a ‘channel’ region, which is a semiconductor path in a substrate. The conductivity of a (typically underlying) channel region in a semiconductor material of the substrate is controlled by the electric field that is generated by the source and the drain. A control electrode, the ‘gate’, is capable of varying this conductivity in that a voltage applied between the gate and source terminals modulates the current between the source and drain terminals. A small change in gate voltage can result in a large change in the current from the source to the drain. A fourth terminal of a FET is the bulk, which may be internally connected to the source. A difference between the voltages of the source and body will change the threshold voltage.

Examples of a FET that may be used in the method of the invention include, but are not limited to, a metal oxide-semiconductor field-effect transistor (MOSFET), including a floating gate MOSFET, a junction field-effect transistor (JFET) or a metal-semiconductor field-effect transistor (MESFET). A MOSFET has a gate electrode of a metal, which is separated from the substrate by an insulating layer (gate dielectric). A respective MOSFET may also be double-gated, such that the metal oxide-semiconductor gate is formed on two, three or four sides of the channel or wrapped around the channel, for example a FinFET.

The formation of an electroconductive metal wire on the nucleic acid capture molecule/target nucleic acid molecule complex may lead to a change in an electronic charge density or a potential, thereby modulating the current between the source and drain of a FET. As already indicated above, the surface of the immobilisation unit on which the capture molecules are immobilised, may be located on or in vicinity to a FET. Generally this surface is or includes the active region of a FET, which is the region from which a signal is detected in response to the formation of a metal wire at the complex formed between the nucleic acid capture molecule and the target nucleic acid molecule (i.e., the analyte molecule). Typically the active region is the area overlaying the portion of the FET that can be influenced by charge or chemical potential. The “active region” is not to be confused with the “active area,” or doped well in which a transistor is defined. The active area of e.g. a MOS transistor equals the product of its channel width and length. In some embodiments the active region of the sensor is at least a part of the gate of a transistor. In some embodiments where a MOSFET is used, the active region may include the insulating layer over the channel region in the absence of a gate electrode. In such embodiments the complex of the nucleic acid capture molecule and the target nucleic acid molecule is completed to form a gate once a metal wire has formed. In some embodiments the active region is located on the floating gate of a field effect transistor or on a semiconductor that is connectively connected to a field effect transistor. In such embodiments the nucleic acid capture molecule may be immobilised on the active region (e.g. the floating gate). The formation of the metal wire may then activate the active region by charge induction. As a result, charge separation in a semiconductor of the active region may occur. Where the active region is conductively connected to the gate of a field effect transistor (see e.g. Krause, M, et al., Sensors and Actuators B (2000) 70, 101-107; US patent application 2006/0029994), the charge may be transferred to this gate. Where the active region is a floating gate the occurring charge may generate a voltage drop between the substrate and the floating-gate, which in turn may activate the field effect transistor. A control gate with the role of a reference electrode may be included in such a FET as described by Barbaro et al. (2006, supra).

As already indicated above, once the immobilisation unit is exposed to an electric field, the metal wire immobilised thereon due to the previous association of the metal ions to the polymer (associated to the complex of the two nucleic acid molecules via the activation agent) is likewise exposed to the respective electric field. This results in an electric signal caused by the metal wire. The metal wire (e.g. nanowire) may for instance change the electric field, change the conductivity or resistance of a medium in the electric field, obtain a charge, transfer charge or conduct a current.

In the method of the invention a signal of the electrically conducting wire may be detected using any detection technique. In this regard any electrical characteristic of a region in the sensing zone may be used for detection purposes as long as the electrical characteristic is influenced by the electrically conducting metal wire. The respective region in the sensing zone may for example be a region in between the electrodes. A detection according to the invention may or instance include a measurement of a conductance, a voltage, a current, a capacitance or a resistance. As an illustrative example, conductance may be measured by linear cyclic voltammetry, square wave voltammetry, normal pulse voltammetry, differential pulse voltammetry and alternating current voltammetry. As a further example already explained above, the immobilisation unit, or at least a part of the surface thereof, may be exposed to an electric field. In this case the wire formed thereon is likewise exposed to the respective electric field. This results in an electric signal. Accordingly, in some embodiments of the method of the invention an electric field is generated, which may in some embodiments be a symmetric or a homogenous electric field. The electric filed may for example be an external field. It may also be generated at least one electrode of the pair of electrodes. If desired, electrochemical impedance spectroscopy may be employed to determine a change in faradaic impedance (see Odenthal & Gooding, 2007, supra, and publications cited therein).

This signal of the electroconductive wire is detected in the method of the present invention. Any detection technique for electric signals may be used in the method of the present invention. A detection according to the invention may or instance include a measurement of a conductance, a voltage, a current, a capacitance or a resistance. As an illustrative example, conductance may be measured by linear cyclic voltammetry, square wave voltammetry, normal pulse voltammetry, differential pulse voltammetry and alternating current voltammetry.

Those skilled in the art will appreciate that the method of the invention is not only rapid and of low cost, but also ultrasensitive and has a high specificity and a high signal to noise ratio. Due to the addition of an activation agent, a polymer and a metal salt dramatic signal amplification is achieved, as can be taken from FIG. 6. In this regard the polymer further contributes to the high sensitivity of the method by providing an increased “catching site” for the metal ions added. Strong chemical interactions can be exploited, such as the ionic binding of metal ions to the polymer and the interaction of the activating agent and the target nucleic acid molecule. The target nucleic acid molecule, i.e. the analyte, may be of any desired length, including a short oligonucleotide. In some embodiments the metal wire is conductively connected to the surface of the immobilisation unit (which may be electrically conductive, see above) or to another surface that is for instance the surface of an electrode. As an illustrative example, the metal wire may bridge across the gap between two electrodes, e.g. of a detection electrode.

Where desired, several different target nucleic acid molecules may be analysed at the same time using either the same immobilisation unit or several immobilisation units in parallel. In embodiments where several target nucleic acid molecules are analysed using the same immobilisation unit, different nucleic acid capture molecules may be immobilised on the immobilisation unit (see above). Where a plurality of each respective capture molecules is used, the number and/or density of each respective capture molecule may be independently selected.

If desired, further methods for detection may be employed. As an example, electroacoustic detection, for instance using a semis-crystalline plastic as described by Gamby et al. (Lab on a Chip (2007) DOI: 10.1039/b707881a) may be employed. As a further example, an optical detection may also be performed or enhanced by means of an optically amplifying conjugated polymer, e.g. in a Förster energy transfer system (Gaylord, B. S., et al., Proc. Natl. Acad. Sci. USA (2005) 102, 34-39; Gaylord, B. S., et al., J. Am. Chem. Soc. (2003) 125, 896-900). Proteins or polypeptides labeled with a fluoresce dye may for instance be used to optically detect their binding to certain target nucleic acid molecules. Nucleic acid intercalating dyes, such as YOYO, JOJO, BOBO, POPO, TOTO, LOLO, SYBR, SYTO, SYTOX, PicoGreen, or Oligreen as available from Molecular Probes, may furthermore be used for optical detection. As a further example, the immobilization unit may include a microcavity, in which the nucleic acid capture molecule is immobilized. It may then define a microtoroid resonator sensor, which may be irradiated and the binding of a target nucleic acid molecule be determined based on the occurring resonance shift (see Armani, A. M., et al., Science (2007) 317, 783-787).

In typical embodiments, the result obtained is then compared to that of a control measurement. In a respective control measurement a nucleic acid capture molecule unable to bind the analyte molecule may for instance be used. An illustrative example of such a “control” capture molecule is a nucleic acid molecule having a sequence not complementary to any portion of the respective target nucleic acid molecule. If the two electrical measurements, i.e. “sample” and “control” measurement, differ in such a way that the difference between the values determined is greater than a pre-defined threshold value, the sample solution contained the relevant analyte molecule.

In some embodiments, the method is designed in such a way that a reference measurement and a measurement for detecting a target nucleic acid molecule are performed simultaneously. This may for instance be done by carrying out a reference measurement only with a control medium and, at the same time, a measurement with the sample solution suspected to contain the analyte molecule to be detected. Likewise, a respective control measurement with a target nucleic acid molecule that has for example comparable properties (e.g. a high degree of sequence identity) but that cannot define a specific binding pair with the nucleic acid capture molecule may be carried out in parallel to a measurement for detecting a target nucleic acid molecule.

The present method also allows detecting more than one target nucleic acid molecule simultaneously or consecutively in a single measurement. For this purpose, a plurality of immobilisation units as described above may for example be used, wherein different types of nucleic acid capture molecules, each of which capable of defining a specific binding pair with a target nucleic acid molecule, are immobilised on each immobilisation unit. Alternatively, a plurality of nucleic acid capture molecules, each of which capable of defining a specific binding pair with a target nucleic acid molecule, may be immobilised on a single surface of an immobilisation unit or on a small number of such surfaces.

The methods according to the present invention may be a diagnostic method for the detection (including quantification) of one or nucleic acids or genes. The analyte molecule may for instance be involved in or associated with a disease or a state of the human or animal body that requires prophylaxis or treatment.

The method of the invention may be combined with other analytical and preparative methods. As already indicated above, the target nucleic acid molecule may in some embodiments for instance be extracted from matter in which it is included. Examples of other methods that may be combined with a method of the present invention include, but are not limited to isoelectric focusing, chromatography methods, electrochromatographic, electrokinetic chromatography and electrophoretic methods. Examples of electrophoretic methods are for instance free flow electrophoresis (FFE), polyacrylamide gel electrophoresis (PAGE), capillary zone or capillary gel electrophoresis. Furthermore the data obtained using the present invention may be used to interact with other methods or devices, for instance to start a signal such as an alarm signal, or to initiate or trigger a further device or method.

The present invention also provides a kit for detecting a target nucleic acid molecule, which may for instance be a diagnostic kit. A respective kit includes a pair of electrodes, which are arranged at a distance from one another, for example separated by a gap. The pair of electrodes is arranged within a sensing zone. A kit according to the present invention furthermore includes an immobilisation unit. The surface of the immobilisation unit is arranged within the sensing zone. As explained above, the sensing zone may for example be defined by the zone in which an electric field of said pair of electrodes is effective.

The kit also includes a nucleic acid capture molecule as described, i.e. with an at least essentially uncharged backbone and with a nucleotide sequence that is at least partially complementary to at least a portion of the target nucleic acid molecule. The kit also includes an activation agent, which has an electrostatic net charge that is complementary to the electrostatic net charge of the target nucleic acid molecule. As explained above, the activation agent may for example be or include a transition metal compound or a metalloid compound and/or may have an affinity to phosphate and/or phosphonate. The kit also includes a water soluble polymer with an electrostatic net charge that is complementary to the electrostatic net charge of the activation agent (see above). Also included in the kit is a metal salt that is capable of acting as an oxidant. The metal ions of the metal salt have an electrostatic net charge that is complementary to the electrostatic net charge of the water soluble polymer (see above). The kit further includes a reducing agent that is capable of reducing the metal ions of the metal salt to the corresponding metal.

A respective kit may furthermore include means for immobilising the nucleic acid capture molecule to the surface of the immobilisation unit. As explained above, a nucleic acid capture molecule included in the kit may have a moiety that allows for, or facilitates, an immobilisation on a respective immobilisation unit. The kit may also include a linking molecule. As an illustrative example, 6-mercapto-1-hexanol may be included in the kit. Where the capture molecule is a nucleic acid molecule, the capture molecule may upon using the kit be 5′-C₆H₁₂SH-modified (see above for examples).

A respective kit may be used to carry out a method according to the present invention. In this regard the kit may include instructions for electrically detecting (including quantifying) a target nucleic acid molecule, for example in form of an instruction leaflet. It may include one or more devices for accommodating the above components before, while carrying out a method of the invention, and thereafter. As an illustrative example, it may include a microelectromedical system (MEMS).

In order that the invention may be readily understood and put into practical effect, particular embodiments will now be described by way of the following non-limiting examples.

### Exemplary Embodiments of the Invention

FIG. 1 depicts a schematic representation of electrical detection of DNA hybridization using gold nanoparticle labels and gold enhancement. Capture DNA (11) is immobilised in the gap between two electrodes (3) (I) and target nucleic acid (12) and gold nanoparticle-labeled detection probe (13) added (II). Hybridization with target DNA and gold nanoparticle-labeled detection probe occurs (III) and a silver salt and a hydroquinone are added (IV). Reductive deposition of silver occurs, creating a bridge that decreases resistance (V).

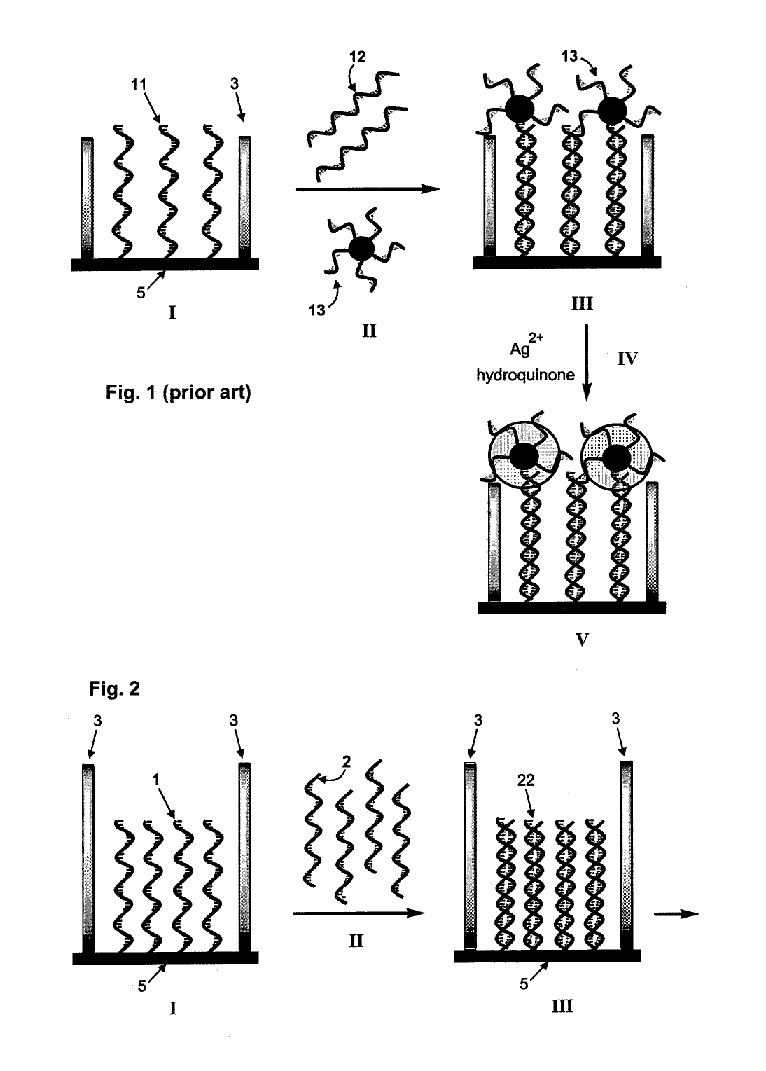

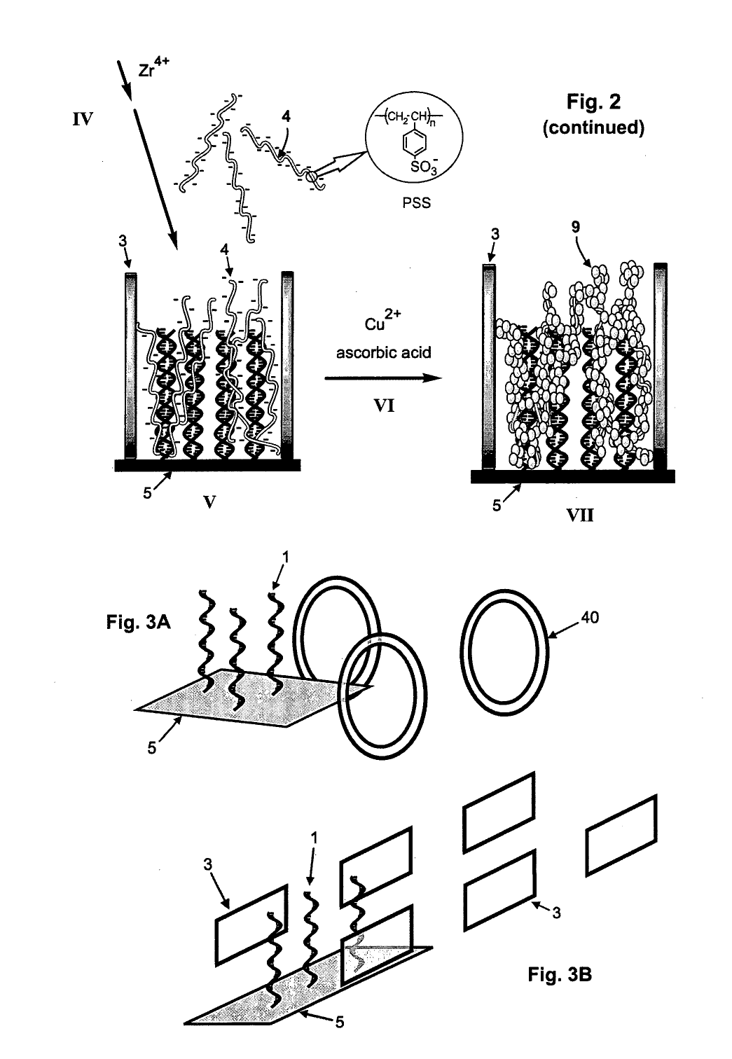

FIG. 2 depicts a schematic representation of a method according to the invention. In the depicted embodiment the immobilisation unit is arranged between two electrodes (3). A nucleic acid capture molecule (1) is immobilised on an immobilisation unit (5) (I). Upon contact with a solution suspected to include the target nucleic acid molecule (2) (II) it forms a complex (22) therewith by hybridization (III). An activation agent (Zr⁴⁺) and a water soluble polymer (4) are added (IV). Polystyrene sulfonate (PSS) is indicated as an exemplary polymer. The polymer associates to the activation agent (V). Addition of a metal salt (Cu²⁺) leads to the association of the metal salt to the polymer, and upon addition of a reducing agent (ascorbic acid) (VI) a metal wire (9) is formed on the complex of the nucleic acid molecules (VII). Based on an electrical characteristic of a region in the sensing zone, which is influenced by the metal wire, the presence of the target nucleic acid molecule can be determined.

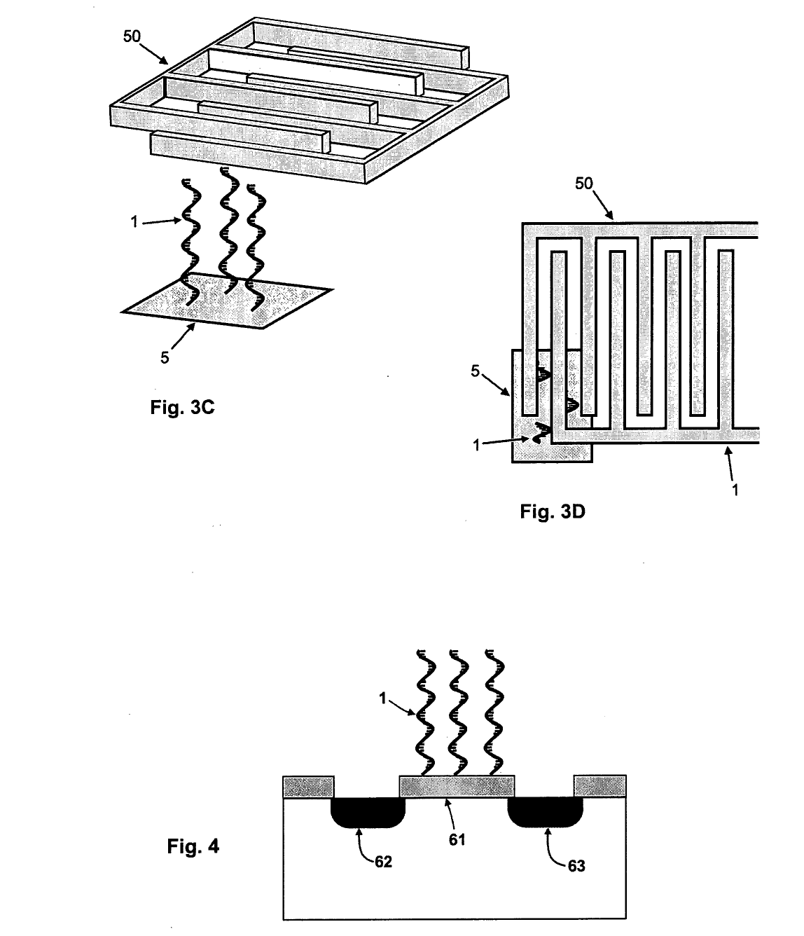

FIG. 3 illustrates examples of an electrode arrangement and an immobilisation unit (5), on which a nucleic acid capture molecule (1) is immobilised. FIG. 3A: Three ring-shaped electrodes (40) are provided. The immobilisation unit (5) is arranged in such a way that its location partly overlaps with the region between two of the electrodes (40). FIG. 3B: An array of electrodes (1) is provided. The array of electrodes (1) defines a region in between them. The immobilisation unit (5) is arranged in vicinity to the electrodes (1), so that the nucleic acid capture molecule (4) is capable of taking an orientation in which its location partly overlaps with the region between two of the electrodes (1). A nucleic acid molecule (not shown) hybridising with the nucleic acid capture molecule (4) will therefore typically take an orientation in which it is essentially located in the region defined by the array of electrodes (1). FIG. 3C: Two interdigital electrodes (50) are provided. The immobilisation unit (5), on which a nucleic acid capture molecule (1) is immobilised, is arranged in vicinity to the electrodes (50) such that the electrical characteristics of the region within electrode areas opposing each other can be influenced by a metal wire associated to the immobilised nucleic acid molecule. FIG. 3D depicts in top view an arrangement of two interdigital electrodes (50) that resembles the arrangement depicted in FIG. 3C. The immobilisation unit (5), on which a nucleic acid capture molecule (1) is immobilised, is arranged below the electrodes (50).

FIG. 4 depicts a schematic representation of a further electrode arrangement that can be used in a method of the invention, in which the nucleic acid capture molecule (1) is immobilised on the gate electrode (61) of a field effect transistor.

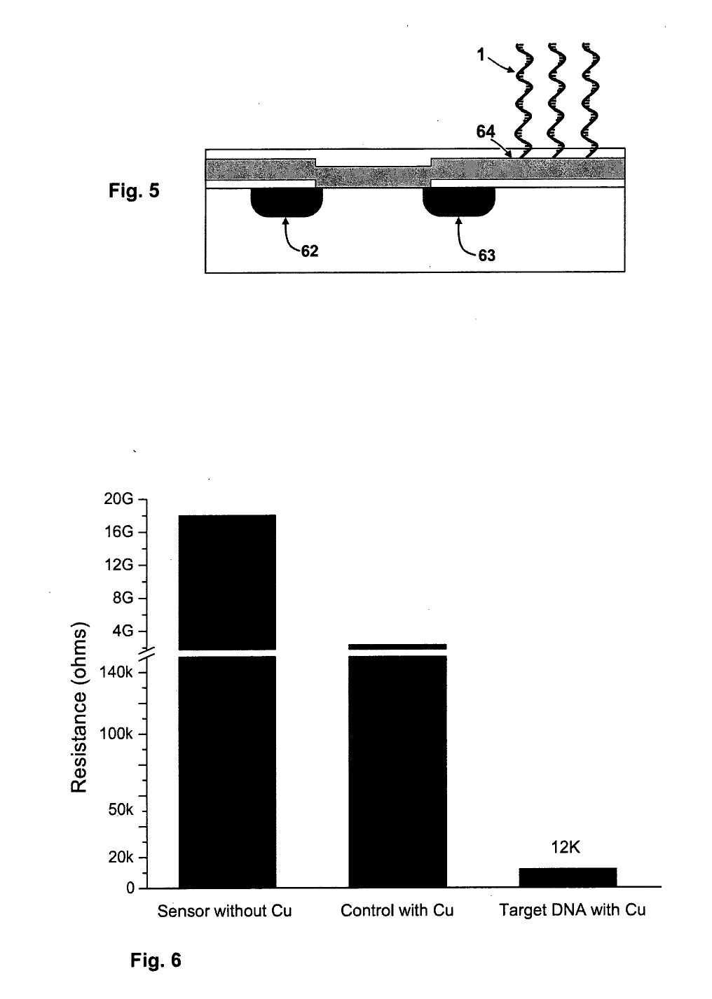

FIG. 5 depicts an electrode arrangement in which the nucleic acid capture molecule (1) is immobilised on an additional, electrically floating gate (64) of a field effect transistor.

FIG. 6 shows the resistance determined of a target nucleic acid being DNA, and a control (indistinguishable) before copper deposition (left), a control after copper deposition (middle) and target DNA after copper deposition (right). Control and/or target DNA (without Cu deposition) were found to be of R=18 GΩ, the control with Cu deposition to be R=2.3 GΩ and target DNA (10⁻¹² M) with Cu deposition to be of R=12 KΩ. It is noted that these data are achieved without any optimisation of the experimental protocol used and that accordingly further improvements in terms of sensitivity can be expected. Indeed, the same nucleic acid as used in experiment the results of which are shown in FIG. 6 was detected at a concentration of 1×10⁻¹³M, 1×10⁻¹⁴M and 1×10⁻¹⁵M as well (data not shown). The experimental protocol used to obtain the results shown in FIG. 6 and the other not shown data were as follows:

Chip preparation and washing: The biosensors that were used in these experiments were fabricated as follows. Chips of a size of 10×10 mm² were fabricated using conventional Silicone (Si) CMOS technology with 500 nm SiO₂ deposited on top of Silicone that was used as substrate. Then gold (Au) interdigitated electrodes were fabricated on the SiO₂/Si substrate using lift-off process for electrical testing. The space and line width of the interdigitated electrodes were 0.5 μm. The so fabricated sensors/chips were soaked in acetone for 20 minutes and then immersed in 99.5% ethanol for 20 minutes, and then finally dried with nitrogen.

Chip silanisation: The silylation reaction of the SiO₂ surface (that acts as immobilization unit) was carried out in an ethanol/water solution as follows. The cleaned substrates were treated with a mixture of ethanol/H₂O/3-aminopropyltriethoxysilane (APTS) (98.5:0.5:1 v/v) for 4 hours. Then the chips/substrates were cleaned with ethanol and acetone and silylated substrates were finally dried for 10 min at 110° C. prior to the further activation.

Chip activation. The chips with its surface being amine-functionalized were reacted for 2 h with 50 mg of 1,4-phenylenediisothiocyanate in 25 ml of a 10% solution of anhydrous pyridine in dimethylformamide. The substrates were subsequently washed with dimethylformamide and dichloromethane before drying under a stream of nitrogen.

Probe immobilization. A PNA probe with the sequence 5′—NH2-AAC CAT ACA ACC TAC TAC CTC A-3′ (SEQ ID NO: 1) was dissolved in 1% trifluoroacetic acid and then diluted to a concentration of 1×10⁻⁵ M in 50 mM Na₂CO₃ and NaHCO₃ buffer (pH 9.0). Spotting substrates were accomplished by adding 100-μl above PNA solution to each substrate. Binding of the amino-functionalized PNA to the activated amine-functionalized surface (of the immobilization unit) was performed over a period of 3 h at 37° C. inside a humid chamber.

Washing and blocking. After spotting the PNA probe of SEQ ID NO: 1 the chips/substrates were rinsed with deionised (DI) water and methanol. The unreacted activated surface of the substrate was deactivated with a solution made of aminoethanol (0.1 ml), diisopropylethylamine (0.65 ml), and dimethylformamide (25 ml) over a period of 2 hours. The slides were subsequently washed with dimethylformamide, acetone, and water, and dried with nitrogen.

Target hybridization. An oligonucleotide with the sequence 5′-TGA GGT AGT AGG TTG TAT GGT T-3′ (SEQ ID NO: 2) was chosen as the target sequence to be detected. Target hybridization was performed at room temperature for 1 hour by using concentrations of the target DNA of 1×10⁻¹²M, 1×10⁻¹³M, 1×10⁻¹⁴M and 1×10⁻¹⁵M in TE buffer. An oligonucleotide with the sequence 5′-GGA AGG GAG TAA AGT TAA TAC CTT TGC TCA TTG ACG-3′ (SEQ ID NO: 3) was used as a negative control with the same concentration.

Posthybridization wash. The substrates were washed with in 1×SSC plus 0.1% SDS for 5 min at room temperature, followed by 0.1×SSC plus 0.1% SDS for 5 min at room temperature. The substrates were rinsed quickly with DI water and then dried with nitrogen.

Zirconium-Phosphate Functionality for Polystyrene Sulfonate. After posthybridization wash, 5 mM ZrOCl₂ in 60% aqueous ethanol solution were added onto the substrates and incubated with this solution for 30 min. The substrates were then rinsed with 70% aqueous ethanol solution, dried with nitrogen add 0.5 mM polystyrene sulfonate (PSS) (molecular weight 70 KDalton, Sigma) solution in H₂O for 30 min washed with 0.1% Igepal CA-630 solution twice for 5 min, and then dried with nitrogen.

PSS-Templated Nanowire Synthesis: 100 μL 0.05 M Cu(NO₃)₂ were added to substrates and the substrates were then incubated in dark for 60 min. The substrates were then rinsed with 70% aqueous ethanol solution, dried with nitrogen and then Cu(NO₃)₂ which was associated with the PNA/target DNA complex via the PPS was reduced by adding 100 μL mixture of 0.05M ascorbic acid solution and 0.05 M Cu(NO₃)₂ to the chip surface for an incubation time of 3.5 minutes. Finally, the substrates were cleaned with Nanopure water (18.2 MΩ), and dried with nitrogen. The resistance of the biosensors (before addition of the copper ions and after the formation of the “nanowires” were measured with an Alessi REL-6100 probe station, together with an ADVANTEST R8340A ultra high resistance meter (an accessory machine which can measure ultra high resistance was used since the resistance measured in these experiments is very high).

The listing or discussion of a previously published document in this specification should not necessarily be taken as an acknowledgement that the document is part of the state of the art or is common general knowledge. All documents listed are hereby incorporated herein by reference in their entirety.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method of electrically detecting a target nucleic acid molecule by means of a pair of electrodes, wherein the electrodes are arranged at a distance from one another and wherein the pair of electrodes is arranged within a sensing zone, the method comprising:
(a) immobilizing on an immobilization unit a nucleic acid capture molecule, wherein the nucleic acid capture molecule has (i) an at least essentially uncharged backbone and (ii) a nucleotide sequence that is at least partially complementary to at least a portion of a strand of the target nucleic acid molecule, wherein the immobilization unit is arranged within the sensing zone;
(b) contacting the immobilization unit with a solution suspected to comprise the target nucleic acid molecule;
(c) allowing the target nucleic acid molecule to hybridize to the nucleic acid capture molecule on the immobilization unit, thereby allowing the formation of a complex between the nucleic acid capture molecule and the target nucleic acid molecule;
(d) adding an activation agent, wherein the activation agent has an electrostatic net charge that is complementary to the electrostatic net charge of the target nucleic acid molecule, such that the activation agent associates to the complex formed between the nucleic acid capture molecule and the target nucleic acid molecule;
(e) adding a water soluble polymer, wherein the water soluble polymer has an electrostatic net charge that is complementary to the electrostatic net charge of the activation agent, thereby allowing the water soluble polymer to associate to the activation agent that is associated to the complex between the nucleic acid capture molecule and the target nucleic acid molecule;
(f) adding a metal salt, wherein the metal salt is capable of acting as an oxidant, the metal ions of the metal salt having an electrostatic net charge that is complementary to the electrostatic net charge of the water soluble polymer, thereby allowing the metal ions to associate to the water soluble polymer, such that a plurality of metal ions covers the surface thereof;
(g) adding a reducing agent, thereby allowing the reducing agent to reduce the metal ions of the metal salt to the corresponding metal, such that a metal wire is formed from the plurality of metal ions covering the surface of the polymer;
(h) determining the presence of the target nucleic acid molecule based on an electrical characteristic of a region in the sensing zone, wherein the electrical characteristic is affected by the electrical conductivity of the metal wire.

2. (canceled)

3. (canceled)

4. (canceled)

5. The method of claim 1, wherein the electrical characteristic affected by the affected by the electrical conductivity of the metal wire is detected by measuring any one of a conductance, a voltage, a current, a capacitance, and a resistance.

6. The method of claim 1, wherein (h) further comprises exposing the immobilization unit to an electric field.

7. The method of claim 6, wherein said electric field is generated at least one electrode of the pair of electrodes.

8. The method of claim 1, wherein the sensing zone is defined by the zone in which an electric field of the pair of electrodes is effective.

9. The method of claim 1, wherein the immobilization unit is arranged in between the pair of electrodes.

10. The method of claim 9, wherein the immobilization unit is arranged in a gap defined by the pair of electrodes.

11. (canceled)

12. (canceled)

13. The method of claim 1, wherein the activation agent is capable of forming a complex with the nucleic acid capture molecule via a coordinative bond.

14. The method of claim 1, wherein the activation agent is a transition metal compound or a metalloid compound.

15. The method of claim 14, wherein the transition metal compound is selected from the group consisting of a zirconium compound, a titanium compound and a niobium compound.

16. (canceled)

17. The method of claim 15, wherein the zirconium compound is zirconyl chloride.

18. The method of claim 1, wherein the target nucleic acid molecule is a DNA molecule or an RNA molecule.

19. The method of claim 1, wherein the nucleic acid capture molecule is one of a peptide nucleic acid (PNA) capture molecule, an alkylphosphonate nucleic acid capture molecule and an alkylphosphotriester nucleic acid capture molecule.

20. The method of claim 1, wherein the nucleic acid capture molecule is single-stranded or comprises a single stranded region.

21. (canceled)

22. (canceled)

23. The method of claim 1, wherein the target nucleic acid molecule comprises a pre-defined sequence.

24. (canceled)

25. (canceled)

26. The method of claim 1, wherein the water soluble polymer is selected from the group consisting of polystyrene sulfonate, poly(vinylsulfate), poly(propenesulfate), poly(butenesulfate), poly(pentenesulfate), poly(hexenesulfate), poly(heptenesulfate), poly(octenesulfate), poly(nonenesulfate), poly(decenesulfate), poly(undecenesulfate), poly(dodecenesulfate), poly(vinylsulfonate), poly(propenesulfonate), poly(butenesulfonate), poly(pentenesulfonate), poly(hexenesulfonate), poly(heptenesulfonate), poly(octenesulfonate), poly(nonenesulfonate), poly(decenesulfonate), poly(undecenesulfonate), poly(vinylphosphate), poly(propenephosphate), poly(butenephosphate), poly(pentenephosphate), poly(hexenephosphate), poly(heptenephosphate), poly(octenephosphate), poly(nonenephosphate), poly(decenephosphate), poly(undecenephosphate), poly(dodecenephosphate), poly(vinylphosphonate), polytpropenephosphonate), poly(butenephosphonate), poly(pentenephosphonate), poly(hexenephosphonate), poly(heptenephosphonate), poly(octenephosphonate), poly(nonenephosphonate), poly(decenephosphonate), poly(undecenephosphonate), poly(dodecenephosphonate), polyglutamate, polyacrylic acid, poly(propylacrylic acid), poly(oxyethylene)diglycolic acid, poly(1-vinyl imidazole), poly(styrene-co-p-vinylbenzoic acid), poly(m-xylene-5-carboxy-m-xylene), poly(2-acrylamido-2-methylpropanesulfonic acid), poly(styrene-co-p-vinylbenzoic acid), poly(2-methoxyaniline-5-sulfonic acid), poly(oxyethylene)diglycolic acid, carboxymethylcellulose, carboxymethyl inulin, an alginate, starch glycolate, a xantham gum, polyglutamate, a hyaluronan, pectin, heparin, and a chondroitin sulfate.

27. The method of claim 1, wherein the water soluble polymer has a molecular weight in the range of about 1000 Da to about 2 million Da.

28. (canceled)

29. The method of claim 1, wherein the metal salt is one of a molybdenum salt, a copper salt, a germanium salt, a tin salt, a rhenium salt an antimony salt, a platinum salt, a palladium salt, a silver salt, a cobalt salt, an iron salt, a bismuth salt and a mercury salt.

30. (canceled)

31. The method of claim 1, wherein the reducing agent is selected from the group consisting of ascorbic acid (vitamin C), oxalic acid, a thiosulphate, thiourea, dithiothreitol, a hydrosulphite, a bisulphite, thioglycolic acid, tertiary butyl hydroquinone (TBHQ), a borane, acetyl cysteine, glucose, a polyphenol compound and poly(vinyl pyrrolidone).

32. (canceled)

33. A kit for electrically detecting a target nucleic acid molecule, the kit comprising:
(a) a pair of electrodes, wherein the electrodes are arranged at a distance from one another and wherein said pair of electrodes is arranged within a sensing zone,
(b) an immobilization unit arranged within the sensing zone;
(c) a nucleic acid capture molecule with (i) an at least essentially uncharged backbone and (ii) a nucleotide sequence that is at least partially complementary to at least a portion of the target nucleic acid molecule;
(d) an activation agent, wherein the activation agent has an electrostatic net charge that is complementary to the electrostatic net charge of the target nucleic acid molecule;
(e) a water soluble polymer, wherein the water soluble polymer has an electrostatic net charge that is complementary to the electrostatic net charge of the activation agent;
(f) a metal salt, wherein the metal salt is capable of acting as an oxidant, and wherein the metal ions of the metal salt have an electrostatic net charge that is complementary to the electrostatic net charge of the water soluble polymer; and
(g) a reducing agent, wherein the reducing agent is capable of reducing the metal ions of the metal salt to the corresponding metal.

34.-43. (canceled)
